(19) United States
(12) Patent Application Publication
(10) Pub. No.: US 2023/0200677 A1
(43) Pub. Date: Jun. 29, 2023

(54) METHODS AND SYSTEMS OF CALIBRATING RESPIRATORY MEASUREMENTS TO DETERMINE FLOW, VENTILATION AND/OR ENDOTYPES

(71) Applicant: NOX MEDICAL EHF, Reykjavik (IS)

(72) Inventors: Sveinbjörn HÖSKULDSSON, Reykjavik (IS); Eysteinn FINNSSON, Reykjavik (IS); Jón Skírnir ÁGÚSTSSON, Reykjavik (IS)

(21) Appl. No.: 18/058,498

(22) Filed: Nov. 23, 2022

Related U.S. Application Data
(60) Provisional application No. 63/282,375, filed on Nov. 23, 2021.

(51) Int. Cl. A61B 5/08; A61B 5/00

(52) U.S. Cl. CPC A61B 5/0806; A61B 5/6823; A61B 5/4818; A61B 5/6803; A61B 5/7246; A61B 5/6831; A61B 2560/0238

(57) ABSTRACT

Methods, systems, and devices are provided for determining a respiratory flow, ventilation, and/or endotypes from Respiratory Inductance Plethysmography (RIP) signals. The method includes receiving data of a thoracic signal of a first RIP belt arranged proximate with a thorax of a subject, receiving data of an abdomen signal of a second RIP belt, and determining a respiratory flow of the subject based on the data of the thoracic signal and the data of the abdomen signal. Determining the respiratory flow includes two or more calibrations, including performing a first calibration by applying a first calibration coefficient that relates an amplitude of a differential change in the thoracic signal to an amplitude of a differential change in the abdomen signal to obtain a determined respiratory flow, and performing a second calibration on the determined respiratory flow that corrects for a non-linearity in the determined respiratory flow.

### CROSS-REFERENCE TO RELATED APPLICATIONS

This application claims the benefit of and priority to U.S. Provisional Patent Application Ser. No. 63/282,375 filed on Nov. 23, 2021 and entitled “Methods and Systems of Calibrating Respiratory Inductance Plethysmography Measurements to Determine Flow, Ventilation and/or Endotypes,” which is expressly incorporated herein by reference.

### FIELD OF THE DISCLOSURE

The present disclosure relates to systems, apparatuses, and methods for performing a personalized sleep study of a subject, and particularly for calibrating respiratory inductance plethysmography (RIP) data, which is then used for endotyping.

### BACKGROUND

Obstructive sleep apnea (OSA) is characterized by transient loss of ventilation during sleep, which promotes arterial hypoxemia and ensuing sympathoexcitation, and has major consequences for daytime health. Reliable clinical estimation of ventilation is important for defining the disorder, and is also important for clinical estimation of advanced olysomnographic measurement of the underlying causes (i.e., endotypic traits). Nasal pressure has been used to semi-quantitatively estimate ventilation, but nasal pressure as a surrogate for ventilation is severely limited due to oral breathing, for example. Accordingly improved techniques are desired for clinical estimation of ventilation. Here, the inventors have developed a novel strategy using respiratory inductance plethysmography (RIP), a measurement that is insensitive to breathing route, to estimate ventilation and the OSA traits.

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### SUMMARY

A method for determining a respiratory flow from Respiratory Inductance Plethysmography (RIP) signals. The method includes obtaining a thoracic signal that corresponds to a length of a first RIP belt arranged proximate with a thorax of a subject, and obtaining an abdomen signal that corresponds to a length of a second RIP belt arranged proximate with an abdomen of the subject. The method further includes determining a respiratory flow based on the thoracic signal and the abdomen signal, wherein the respiratory flow is determined using two or more calibrations. The two or more calibrations include a first calibration applying a first calibration coefficient that relates an amplitude of a differential change in the thoracic signal to an amplitude of a differential change in the abdomen signal. And the two or more calibrations include a second calibration that corrects for a non-linearity in the determined respiratory flow.

### BRIEF DESCRIPTION OF THE DRAWINGS

FIG. 1 shows a flow diagram of a method of endotyping sleep disorders based on calibrated Respiratory Inductance Plethysmography (RIP) data;

FIGS. 2A, 2B, and 2C illustrate an example of respiratory inductance plethysmograph (RIP) belts, 2A shows an example of the wave-shaped conductors in the belts, 2B shows the cross-sectional area of each belt, which is proportional to the measured inductance, and FIG. 2C illustrates an embodiment of an RIP belt;

FIG. 2D illustrates a model of volumes for a thorax and an abdomen;

FIG. 3 illustrates a PolySomnoGraphy (PSG) setup;

FIG. 4 illustrates a simplified PolySomnoGraphy (PSG) setup;

FIG. 5 illustrates measured RIP traces;

FIG. 6 illustrates derived RIP volumes;

FIG. 7A illustrates derived RIP traces;

FIG. 7B illustrates derived RIP flows compared to pneumotach flows;

FIG. 7C illustrates a curve fit for an overestimation correction factor (OCF);

FIG. 7D illustrates derived RIP flows corrected according to the OCF;

FIG. 8 illustrates an alternative curve fit for an overestimation correction factor (OCF);

FIG. 9 illustrates a derivation of the flow and ventilation values from RIP traces;

FIG. 10A illustrates a Continuous Positive Airway Pressure (CPAP) maneuver;

FIG. 10B illustrates the ventilatory response to changes in CPAP pressure;

FIG. 11 illustrates the eupneic ventilation;

FIG. 12 illustrates a ventilation trace and scored arousals from a simulation of a respiratory model fit as an example of OSA endotyping;

FIG. 13 illustrates a flow diagram of a process to validate RIP-based endotyping.

FIG. 14 illustrates statistics for RIP ventilation with and without applying the OCF, showing respective biases in minute ventilation with and without OCF;

FIGS. 15A and 15B illustrate ventilation error without a non-linearity correction;

FIGS. 16A and 16B illustrate ventilation error with a non-linearity correction;

FIG. 17 illustrates a comparison of experimental results comparing event depth for pneumotach and RIP flow using a fixed calibration constant;

FIG. 18 illustrates a comparison of experimental results comparing event depth for pneumotach and RIP flow using a calibration constant;

FIG. 19A illustrates a comparison of experimental results comparing arousal threshold for pneumotach and RIP-based flows, respectively;

FIG. 19B illustrates a comparison of experimental results comparing upper airway collapsibility (Vpassive) for pneumotach and RIP-based flows, respectively;

FIG. 19C illustrates a comparison of experimental results comparing loop gain for pneumotach and RIP-based flows, respectively;

FIG. 19D illustrates a comparison of experimental results comparing ventilation at the arousal threshold for pneumotach and RIP-based flows, respectively;

FIG. 19E illustrates a comparison of experimental results comparing upper airway muscle compensation for pneumotach and RIP-based flows, respectively;

FIG. 20 illustrates intra-class correlation (ICC) values for RIP-based endotyping;

FIG. 21 illustrates flow traces for pneumotach, nasal canula, and RIP flows as an example of the effects of oral ventilation; and

FIG. 22 illustrates schematic diagram for a device that performs the method illustrated in FIG. 1;

FIG. 23A illustrates a plot comparing event depth in terms of the percent ventilation for normal breathing (eupnea) for pneumotach ventilation compared to RIP-based ventilation;

FIG. 23B illustrates a plot of pneumotach ventilation compared to ventilation based on nasal cannula measurements;

FIG. 24 illustrates ventilation as a function of time exhibiting a cycle of hypoventilation (below eupnea) followed by hyperventilation (above eupnea); and

FIG. 25 illustrates a conversion of flow values to ventilation values, which exhibit periods of hypopnea and apnea.

### DETAILED DESCRIPTION OF VARIOUS EMBODIMENTS

An object of the present application is to provide methods, systems and apparatuses that increase the accuracy of estimates of ventilation based on respiratory inductance plethysmography (RIP) measurements. The accuracy of the ventilation estimates is improved through a series of calibration as described below. These calibrations include, without limitation to order, (1) determining a period during which the RIP-belts calibration is constant, (2) determining a ratio (k) between the abdominal and thoracic volumes, (3) calibrating an overestimation correction factor (OCF) for RIP paradox overestimation, and calibrating a correction for RIP non-linearity. The estimates of ventilation may then be used in predictions of endotyping and/or phenotyping based on respiratory inductance plethysmography (RIP) measurements. The endotyping and/or phenotyping are rendered more accurate by the increase in accuracy in the estimates of ventilation. As stated above, this increase in accuracy derives from improvements in methods of calibrating the respiratory inductance plethysmography (RIP) measurements, including, e.g., calibrations to determine the ratio of abdominal to thoracic volume, calibration correcting for RIP paradox overestimation, and calibration correcting for RIP non-linearity.

The RIP measurements may be obtained from Simplified Sleep Studies (SSS). SSS may serve as valuable tools for monitoring sleep or care management of individuals being treated for different medical disorders, and particularly to provide methods, systems and apparatuses with increased the accuracy. The SSS may be performed using devices applied by the patient himself without requiring nightly or regular professional assistance.

Obstructive sleep apnea (OSA) endotyping is a method for identifying the pathophysiological traits that contribute to obstructive sleep apnea (OSA). These traits may include upper airway collapsibility, upper airway muscle compensation, arousal threshold, and loop gain, as discussed in A. Wellman et al., “A method for measuring and modeling the physiological traits causing obstructive sleep apnea,” Journal of Applied Physiology, vol. 110, no. 6, pp. 1627-1637, June 2011, which is incorporated herein in its entirety.

Understanding the pathogenesis of OSA for individual patients can be used for targeted treatment. The methods described herein provide more accurate endotyping for the pathogenesis of OSA based on respiratory inductance plethysmography (RIP) measurements, and this more accurate endotyping may be used as an integral part of precision OSA diagnosis and treatment, as discussed in W. Randerath et al., “Challenges and perspectives in obstructive sleep apnea: Report by an ad hoc working group of the Sleep Disordered Breathing Group of the European Respiratory Society and the European Sleep Research Society,” Eur Respir J, vol. 52, no. 3, p. 1702616, September 2018, which is incorporated herein in its entirety.

In certain non-limiting embodiments described herein, respiratory inductance plethysmography (RIP) calibration is applied to minute ventilation and OSA endotyping by calibrating the RIP data before applying it the method described in E. Finnsson et al., “A scalable method of determining physiological endotypes of sleep apnea from a polysomnographic sleep study,” Sleep, vol. 44, no. 1, p. zsaa168, January 2021, which is incorporated herein in its entirety.

OSA endotypes are derived from patients' breathing patterns. Previously, patients' breathing patterns were captured with flow sensors, but flow sensors have various drawbacks. For example, flow measurements via nasal canula fail to capture ventilation arising from mouth breathing, which may be significant for OSA, and pneumotach with oronasal mask, which do captures ventilation due to both nose and mouth breathing, is not readily available in clinical settings, for various reasons. Accordingly, for improved ventilation estimation and endotyping in clinical settings, other sensors are needed to better capture ventilation due to both nose and mouth breathing. The methods described herein address this issue using RIP measurements which determine the ventilation indirectly through changes in abdominal and thoracic volumes.

In certain embodiments, RIP measurements are used to determine the amount of ventilation during each minute of sleep (minute ventilation), as described in S. Chokroverty, “Sleep and breathing in neuromuscular disorders,” in Handbook of Clinical Neurology, vol. 99, Elsevier, 2011, pp. 1087-1108, which is incorporated herein in its entirety. And the minute ventilation is the basis for endotype determination.

Using minute ventilation for endotype determination has been validated in laboratory settings using pneumotach with a sealed oronasal mask, as described in A. Wellman et al., “A method for measuring and modeling the physiological traits causing obstructive sleep apnea,” Journal of Applied Physiology, vol. 110, no. 6, pp. 1627-1637, June 2011; and in S. A. Sands et al., “Phenotyping Pharyngeal Pathophysiology using Polysomnography in Patients with Obstructive Sleep Apnea,” Am J Respir Crit Care Med, vol. 197, no. 9, pp. 1187-1197, May 2018, which are both incorporated herein in their entirety. When used with an oronasal mask, the pneumotach measures unbiased flow and circumvents possible confounding effects of oral ventilation. Ventilation can be either nasal or oral. Polysomnographic (PSG) sleep studies that solely use a nasal flow sensor (nasal cannula) fail to measure oral ventilation. Accordingly, PSG sleep studies based on pneumotach with oronasal mask that measure airflow through both breathing routes are superior. This is an important advantage, as patients with OSA spend a significantly larger proportion of the night in oronasal breathing than those without OSA, who are more likely to be nasal breathers. Despite this, the pneumotach with oronasal mask is not readily available in clinical settings which limits the clinical applications of endotyping. Therefore, the methods described herein advantageously provide endotyping based on data that is readily available in clinical settings by using RIP data for endotyping, such that the method can be applied to standard PSG recordings.

Discussed herein is experimental data demonstrating the reliability of the method of endotyping based on RIP data without using other flow data. The experimental data shows the results of applying endotyping to standard PSG studies (i.e., RIP data), by validating the performance of the methods described herein through comparisons to endotypes gathered from PSG flow sensors to endotypes from the gold standard oronasal pneumotach. In a PSG sleep study, a nasal cannula is used for measuring nasal ventilation.

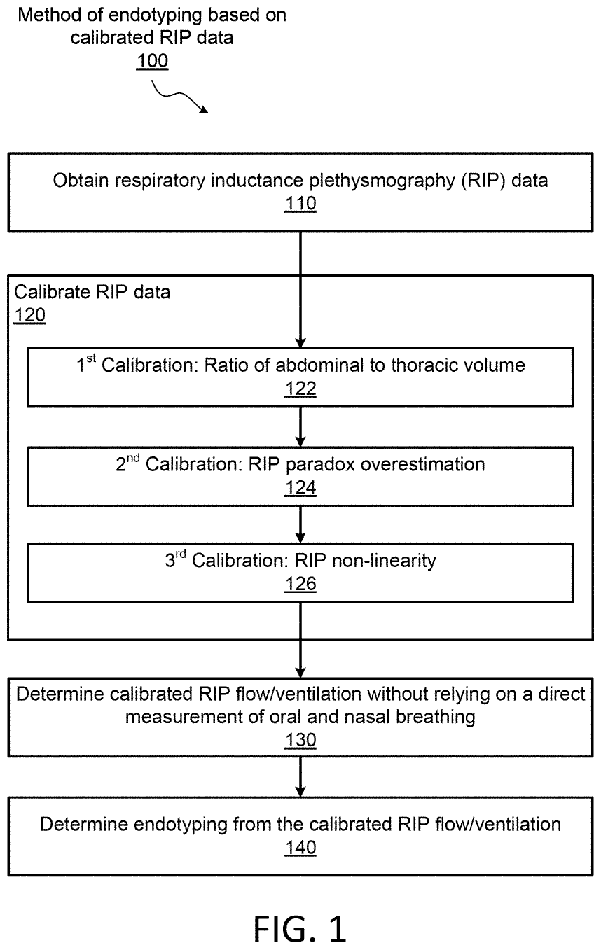

Referring now to FIG. 1, a method is illustrated for determining OSA endotypes based on the RIP data. There are several advantages to using RIP measurements, rather than nasal canula, to determine ventilation and endotyping. Even if it were feasible to use nasal cannula to detect a subset of the OSA endotypes, the nasal cannula may become dislodged during sleep, resulting in a lack of signal. Further, validating nasal cannula for the detection of OSA endotypes is challenging due to the difficulties of simultaneous pneumotach and nasal pressure measurements and the possibility of one measurement influencing the other. In contrast, pneumotach and RIP measurements are straightforward to perform simultaneously because they are respectively arranged on separate parts of the body (i.e., the head for pneumotach measurements and the chest and abdomen for RIP measurements). That is, the pneumotach/mask does not interfere with RIP measurements, so simultaneous measurement requires no adjusted setup.

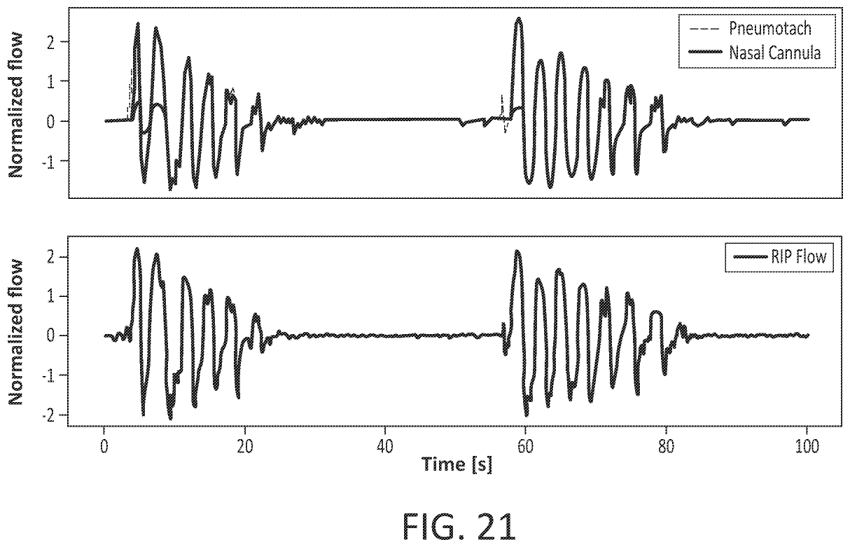

Referring now to FIG. 21 illustrates the relative advantages of using RIP measurements to predict ventilation, instead of using nasal cannula measurements. The upper plot of FIG. 21 shows the flow as a function of time for pneumotach measurements and for nasal cannula measurements. The plots show an apnea in which the flow decreases to zero for a time period and then the patient starts breathing again. During the recover breath when the patient starts breathing again a significant amount of the flow is due to oral breathing, which is not measured by the nasal cannula. In contrast, the predicted by the RIP measurements, which is shown in the lower plot of FIG. 21, closely matches the pneumotach flow, even for the recovery breathes. As can be seen, it is important to account for mouth breathing, therefore the determination of RIP flow is a significant contribution. Oral ventilation is especially important for OSA endotyping. As illustrated in FIG. 21, positive deflections are inhalations and negative deflections are exhalations. When the line flatlines, the patient has an apnea. In the upper plot, the dark grey line is measured using an oro-nasal mask while the light grey line is measured using a nasal cannula. The nasal cannula is typically used for a standard sleep study, while the pneumotach (oro-nasal) is difficult to apply at mass and therefore is not used for a standard sleep study. In the lower plot, the grey line is the RIP flow which is not sensitive to the breathing route. The methods described herein use RIP belts to provide a very accurate estimation of the oro-nasal flow.

There are several more advantages for using RIP measurements to estimate ventilation and endotyping. An airflow estimate can be derived from respiratory inductance plethysmography (RIP), which provides a surrogate measure of breathing through thoracic and abdominal movement. RIP is part of a standard PSG recording and is recommended by the American Academy of Sleep Medicine (AASM) for monitoring respiratory effort and as a secondary airflow sensor. With proper measurement and signal processing, RIP can be used to accurately and reliably measure ventilation independent of breathing route, as discussed in K. Montazeri, S. A. Jonsson, J. S. Agustsson, M. Serwatko, T. Gislason, and E. S. Arnardottir, “The design of RIP belts impacts the reliability and quality of the measured respiratory signals,” Sleep Breath, vol. 25, no. 3, pp. 1535-1541, September 2021, which is incorporated herein in its entirety. Successful validation of endotyping from RIP data has the advantage of making OSA endotyping easily available in the clinical setting. An integral step in this methodology is the calibration of the RIP signal.

Returning to FIG. 1, a non-limiting flow diagram is illustrated of a method 100 for endotyping based on calibrated RIP data. In step 110 of method 100, RIP data is obtained. In step 110, RIP belts measure an inductance from which changes in the length of the belts can be determined. Additionally, the constant (e.g., DC) signals from the belts can be used to determine the total length of the belts. The lengths of the RIP belts are related to the circumferences of the chest (Thorax) and abdomen, from which can be determined the thoracic and abdominal volumes and by extension the ventilation. However, to more accurately determine the ventilation, several calibrations are needed.

In step 120 of method 100, three calibrations are performed, although the order of these calibrations may differ in respective embodiments. In a first calibration 122, a ratio (k) is determined between the abdominal to thoracic volumes. To make this first calibration 122 more accurate a preliminary step may be taken to determine periods over which the ratio k does not change. For example, the position of the RIP belts may change throughout the night due to the patient tossing and turning in their sleep. These changes may be detected by monitoring the DC signal from the RIP belts. In a second calibration 124, a correction is made accounting for RIP paradox overestimation, as discussed below. In a third calibration 126, a correction is made accounting for RIP nonlinearity, as discussed below.

In step 130 of method 100, the above-noted celebrations are applied to the measurements from the RIP belts to generate ventilation estimates. In certain embodiments, the ventilation estimates are generated in the absence of flow measurements.

In step 140 of method 100, the ventilation estimates are used for endotyping and/or phenotyping.

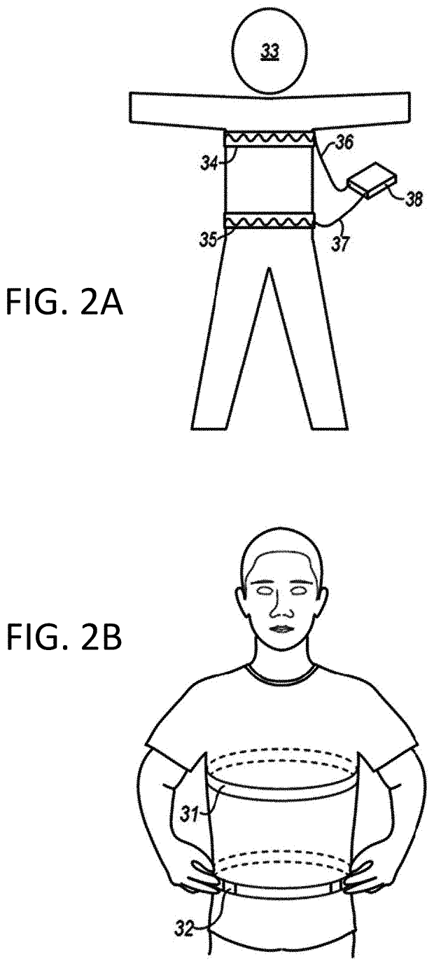

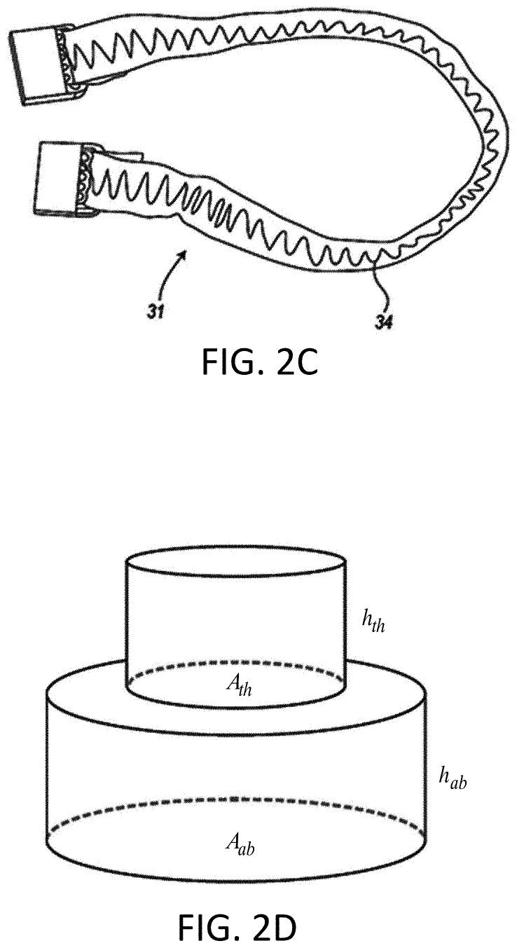

FIGS. 2A-2D illustrate an embodiment of the RIP belts. In the example of RIP, sensor belts may be capable of measuring either changes in the band stretching or the area of the body encircled by the belt when placed around a subject's body. A first belt may be placed around the thorax and second belt may be placed around the abdomen to capture respiratory movements caused by both the diaphragm and the intercostal-muscles. The area that the belt encircle can be used to derive volume and change in volume is flow

Volume=area_abdomen*height_abdomen+area_thorax*height_thorax

Flow=Δarea_abdomen*height_abdomen+Δarea_thorax*height_thorax

When sensors measuring only the stretching of the belts are used, the resulting signal is a qualitative measure of the respiratory movement. This type of measurement is used, for example, for measurement of sleep disordered breathing and may distinguish between reduced respiration caused by obstruction in the upper airway (obstructive apnea), where there can be considerable respiratory movement measured, or if it is caused by reduced effort (central apnea), where reduction in flow and reduction in the belt movement occur at the same time.

Unlike the stretch-sensitive respiratory effort belts, areal sensitive respiratory effort belts provide detailed information on the actual form, shape and amplitude of the respiration taking place. If the areal changes of both the thorax and abdomen are known, by using a certain calibration technology, the continuous respiratory volume can be measured from those signals and therefore the respiratory flow can be derived.

Respiratory Inductive Plethysmography (RIP) is a method to measure respiratory related areal changes. As shown in FIGS. 2A, 2B, and 2C, in RIP, stretchable belts 31, 32 may contain a conductor 34, 35 that when put on a subject 33, form a conductive loop that creates an inductance that is directly proportional to the absolute cross-sectional area of the body part that is encircled by the loop. When such a belt is placed around the abdomen or thorax, the cross-sectional area is modulated with the respiratory movements and therefore also the inductance of the belt. Conductors 34, 35 may be connected to signal processor 38 by leads 36, 37. Processor 38 may include a memory storage. By measuring the belt inductance, a value is obtained that is modulated directly proportional with the respiratory movements. RIP technology includes therefore an inductance measurement of conductive belts that encircle the thorax and abdomen of a subject.

In another embodiment, conductors may be connected to a transmission unit that transmits respiratory signals, for example raw unprocessed respiratory signals, or semi-processed signals, from conductors to processing unit. Respiratory signals or respiratory signal data may be transmitted to the processor by hardwire, wireless, or by other means of signal transmission.

Resonance circuitry may be used for measuring the inductance and inductance change of the belt. In a resonance circuit, an inductance L and capacitance C can be connected together in parallel. With a fully charged capacitor C connected to the inductance L, the signal measured over the circuitry would swing in a damped harmonic oscillation with the following frequency:

f=1/(2π√LC),

until the energy of the capacitor is fully lost in the circuit's electrical resistance. By adding to the circuit an inverting amplifier, the oscillation can however be maintained at a frequency close to the resonance frequency. With a known capacitance C, the inductance L can be calculated by measuring the frequency f and thereby an estimation of the cross-sectional area can be derived.

The RIP data may be obtained while performing a High-Accuracy Sleep Study (HASS), while performing a Simplified Sleep Study (SSS), or any other sleep study lying along the spectrum of sleep-study accuracy and including the acquisition of RIP belt measurements.

In certain Simplified Sleep Studies (SSS), a patient can perform the hookup himself. Simplified Sleep Studies (SSS) have been developed both as medical devices and as commercial devices made available to consumers.

In the SSS medical devices the simplification may result from simplifying the PSG sleep study by recording a subset of the PSG signals. Reducing the number of signals has however a limiting effect on the outcome. The performance and accuracy of the SSS sleep profile is reduced. SSS methods generally only work on a limited group of subjects and the accuracy and performance is especially reduced when used by those having sleep disorders. For clinical purposes, SSS are however often used for specific sleep disorders, such as to confirm sleep apnea, where reasonable accuracy can be achieved based on the measure of respiratory and oximetry parameters only, allowing the EEG, EOG, ECG and EMG signals to be skipped. Further reduced sleep studies, such as those based on oximetry only, are also practiced with further reduced accuracy and mostly used for screening purposes only.

Along the sleep study spectrum of accuracy, performance, or precision, a standard HASS may be more accurate than what may be termed a “practical” HASS, the practical HASS still providing high accuracy when compared to, for example, a Simple Sleep Study (SSS). In an embodiment, a practical HASS method (as compared to a Standard HASS or PSG) may be based on a Self Applied Somnography (SAS). SAS is a sleep study that is designed to provide close to the same information and performance for sleep profiling as standard PSG or Standard HASS but has the benefit that most people can successfully place the sensors on themselves and perform the recording. This allows the equipment and sensors to be delivered over the counter or by mail to the patient that can then perform the PSC calibration study himself before returning the SAS system to the clinic or shipping it back over mail.

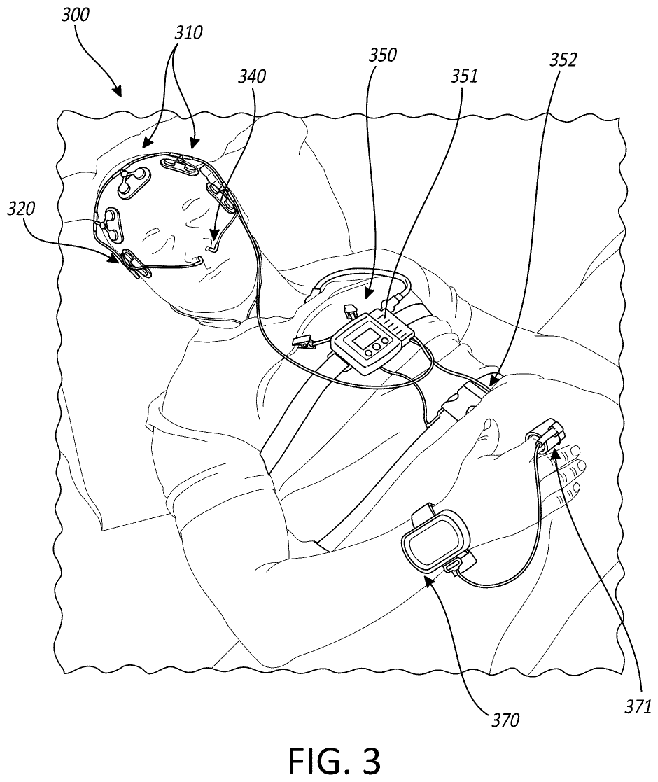

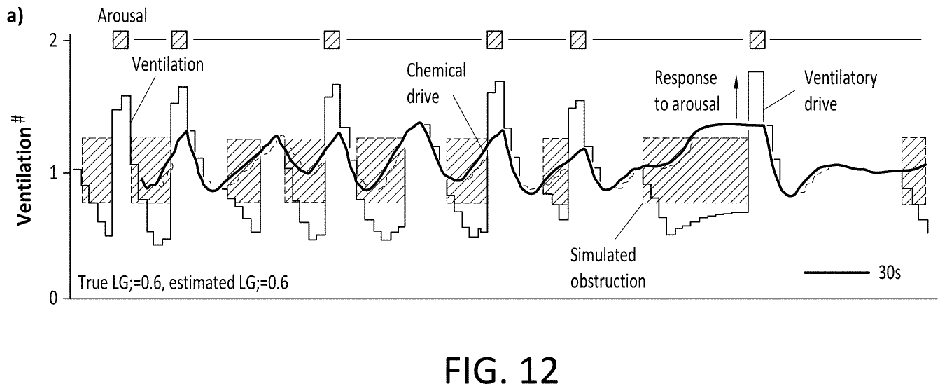

FIG. 3 shows a subject undergoing such a practical HASS according to this embodiment, in the form of a SAS. As shown in FIG. 12 a subject 300 may preferably apply the following sensors and devices to himself, or an untrained or uncertified assistant, such as a family member, roommate, or untrained or uncertified medical work. EEG electrodes 310 may be attached to the head of the subject. The electrodes 310 may be arranged in a band to ensure proper placement. The band may also include, but does not necessarily include, EOG electrodes 320 placed on one or more distal ends of the headband so as to be arranged near an eye of the subject 300. Furthermore, the patient 300 may have a nasal cannula 340 used to measure nasal breathing. The subject 300 may also have respiratory inductance plethysmography (RIP) stretchable belts 351, 352 placed around his chest (thoracic) and abdomen, respectively, to measure breathing movements. Stretchable belts 351,352 may contain a conductor (not shown) that when put on a subject 300, form a conductive loop that creates an inductance that is directly proportional to the absolute cross sectional area of the body part that is encircled by the loop. When such a belt is placed around the abdomen or thorax, the cross-sectional area is modulated with the respiratory movements and therefore also the inductance of the belt. Conductors in the belts may be connected to signal processor 350 by leads or transmitted or received by the processor 350 wirelessly. Processor 350 may include a memory storage. By measuring the belt inductance, a value is obtained that is modulated directly proportional with the respiratory movements. RIP technology includes therefore an inductance measurement of conductive belts that encircle the thorax and abdomen of a subject.

The subject 300 of the embodiment of FIG. 12 may also have a pulse oximeter 370 on the wrist and a corresponding sensor 371 on a finger, such as an index finger, to measure the blood oxygen saturation and pulse in the finger. Furthermore, the patient may also have leg EMG leads although not shown in FIG. 12.

In another embodiment, an advanced SAS system may be used that is based on the use of wireless Smart Sensors that minimize the use of cables and further simplify significantly the hookup of the sensors while maintaining the performance and high-resolution of the cabled SAS or PSG methods. Such an advanced SAS is also preferable in that movement or turning of the subject during the study is not encumbered by wires or cables.

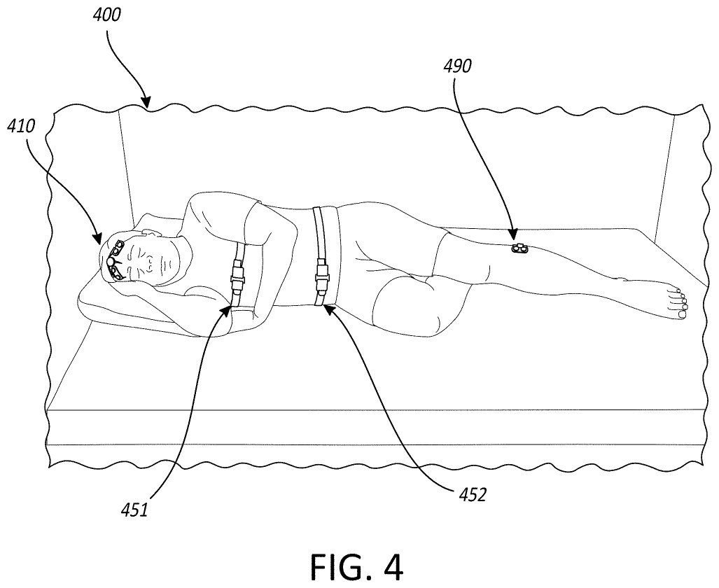

FIG. 4 shows a schematic of a subject 400 sleeping with the wireless SAS sleep study. The devices worn by the subject in such as study may include EEG electrodes 410 placed on the forehead of the subject, respiratory inductance plethysmography (RIP) belts 451, 452, and a sensor or leg EMG lead 490 applied to the leg of the subject. As noted above, these sensors and devices have been provided to the subject in such a way that the subject can easily and consistently apply the sensors and devices himself (or herself).

Returning to FIG. 1, after step 110 in which the data is acquired via the RIP belts, the RIP data is calibrated in step 120 using one or more calibrations. In a first calibration, a ratio is determined between the abdominal volume and the thoracic volume.

The first RIP calibration is intuitively understood by considering that relative changes in lung volumes can be estimated by measuring the diameter of the thorax and abdomen and adding them together in the right proportions (e.g., summing them using a proportionality constant “k”). The proportionality constant can be found by performing an isovolume breathing maneuver, inducing paradoxical movement of the abdomen and thorax, and choosing the calibration constant (k) such that the measured amplitudes of the abdominal and thoracic excursions cancel out. This is called Isovolume maneuver calibration (ISOCAL), as discussed in K. Konno and J. Mead, “Measurement of the separate volume changes of rib cage and abdomen during breathing,” Journal of Applied Physiology, vol. 22, no. 3, pp. 407-422, March 1967, which is incorporated herein in its entirety.

In a PSG sleep study, the calibration constant may change throughout the night. For example, the sleeping subject may move throughout the night, displacing the belts move from their original position. Consequently, when breathing movements are recorded using RIP belts, ISOCAL calibration may be challenging. To overcome this challenge, the calibration constant may be updated throughout the study by inferring the calibration constant (k) from the signals resulting from spontaneous breathing throughout the night. For example, qualitative diagnostic calibration (QDC) may be used to recalibrate the calibration constant (k) throughout the study. In certain embodiments, the calibration constant (k) is chosen such that the variance between the abdominal and thoracic tidal volumes is normalized over a moving, non-overlapping, 5 minute window.

Alternatively, the calibration constant (k) may be chosen such that the two RIP belts are simply added together in a fixed ratio of 2:1 (chest-to-abdomen).

The RIP bands measure changes in the circumference of the thorax and abdomen. Assuming a constant height of these two compartments, the RIP signals may be used to infer respective volumes (or changes in volume) for the thorax and abdomen. A flow estimate can be calculated from the RIP signals by using the time derivative of the calibrated RIP signal according to the following equation

RIP_flow=kdRIP_Ab+(1−k)dRIP_Th,

wherein dRIP_Ab and dRIP_Th are respectively time derivatives of the abdominal and thoracic RIP signals, and k is the calibration constant.

Returning to FIG. 1, a first calibration is performed to generate the calibration constant k, which relates the abdominal and thoracic volumes. To calibrate the belts, only information available from the RIP signals themselves is used. For example, this calibration does not depend on any (potentially unavailable) airflow signals. Advantageously, this calibration may be performed across the night in the event of overnight changes. The calibration of the calibration constant k may be performed using the “power loss” method, which aims to find the optimal calibration constant k (where the ‘calibrated’ tidal volume signal=[1−k]RIPₜₕ+[k]RIP_{ab}; RIPₜₕ and RIP_{ab} are the thoracic and abdominal volume signals respectively). Optimal k is found by minimizing the ratio of the root mean square (RMS) of the ‘calibrated’ flow signal (derivative of the volume signal) to the sum of the RMS of the separate thoracic and abdominal flow signals, i.e.:

$$\frac{R M S ( [ 1 - k ] \frac{d}{dt} {RIP}_{t h} + [ k ] \frac{d}{dt} {RIP}_{a b} )}{R M S ( [ 1 - k ] \frac{d}{dt} {RIP}_{t h} ) + R M S ( [ k ] \frac{d}{dt} {RIP}_{a b} )}$$

In principle, when the calibration constant is tuned appropriately, the paradox component seen in each separate signal ([1−k]RIPₜₕ, [k]RIP_{ab}) is maximally subtracted out in the calibrated sum. In certain embodiments, recalibration is performed when a patient moves. For example, k may be updated using an adaptive strategy whereby the night is subdivided into multiple constant calibration periods (typically >5 minutes) separated by automatically-detected movement events. The calibration constant k may be maintained constant within each period, thereby mitigating the adverse effects of non-constant tidal volume on RIP calibration.

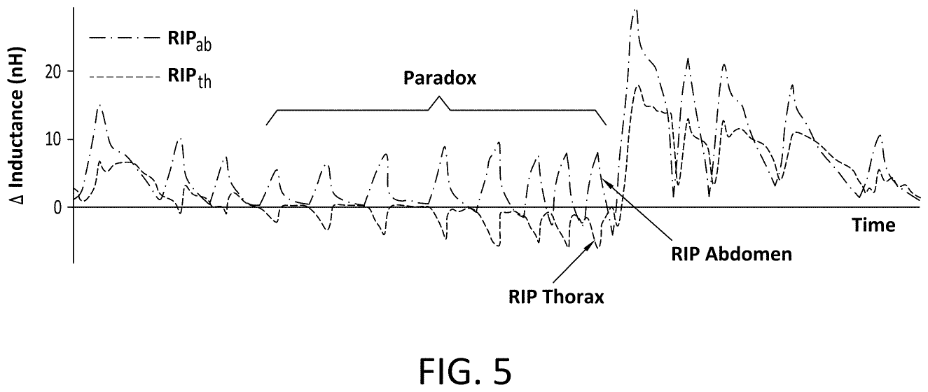

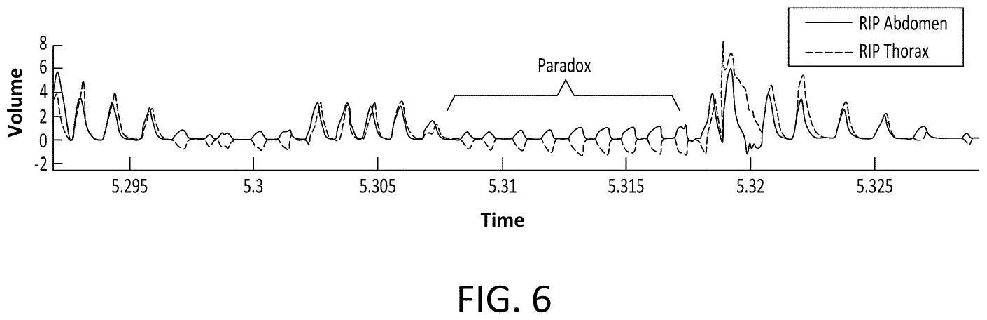

FIGS. 5 and 6 illustrate RIP measurements during an apnea. FIG. 5 illustrates the raw measurements of the inductance of the RIP belts, and FIG. 5 illustrates the volumes estimated from other measurements of the inductance of the RIP belts. The illustrated behavior is what one would expect during an apnea. The upper airway resistance increases during the apneas, as indicated by the out of phase movements of the RIP signals, which is indicated as paradox (i.e., paradoxical breathing. During this period there is no flow/ventilation and the total volume remains constant. Accordingly, like during an ISOCAL calibration, the signal during this period may be used to calibrate the calibration constant k.

The calibration constant k may change throughout a sleep study due to displacement of the RIP belts from their original locations. This can be monitored using a DC signal from the RIP belts. For example, changes to the DC signal indicate that the averaged length of the belt has changed, and a new value for the calibration constant k. Thus, the calibration constant k may take on a series of discrete values in respective constant calibration periods throughout the sleep study. Each period of constant calibration is determined by monitoring for changes in the DC signals from one or more of the RIP belts. In each constant calibration period, the calibration constant k may be determined using the above calibration process, for example, to have the volumes for the RIP abdomen and RIP thorax effectively cancel each other during the period of paradox.

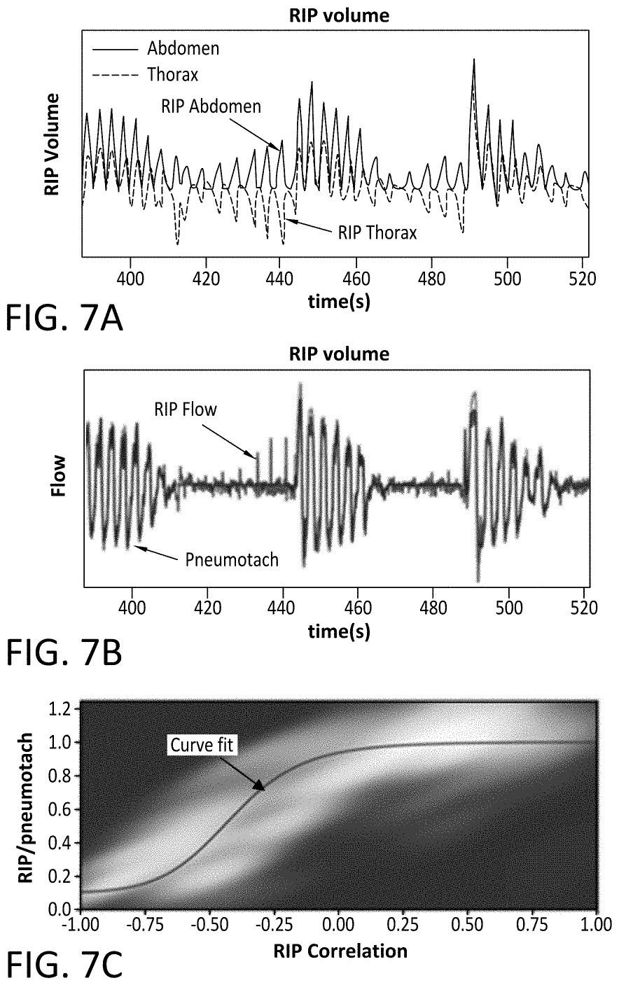

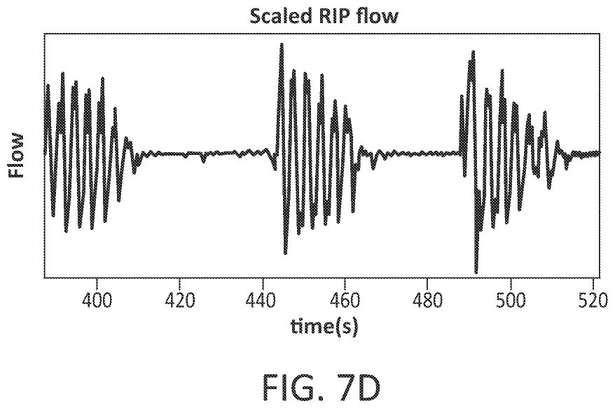

Returning to FIG. 1, a second calibration 124 is performed to determine and correct for RIP paradox overestimation. Thoracic and abdominal excursions describe just two compartments of a more complex respiratory system. Accordingly, even the optimal value of the calibration constant k does not result in the paradoxical movements being perfectly cancelled. Consequently, there is a systematic overestimation of ventilation during paradox. This overestimation is illustrated in FIGS. 7A and 7B. FIG. 7A shows the calibrated RIP volumes for the abdomen and the thorax. During paradox, these two signals are clearly out of phase (i.e., the peaks in one signal occur simultaneously with the troughs from the other). FIG. 7B illustrates (dark grey line) the RIP flow generated using the calibrated RIP volumes. Further, FIG. 7B illustrates the pneumotach flow (black line), which unlike the RIP flow goes to zero during paradox. Thus, compared to the pneumotach flow, the RIP flow overestimates the flow/ventilation during paradox.

To correct this overestimation, the amount of overestimation may be determined using the curve fit shown in FIG. 7C. In FIG. 7C, the horizontal axis is the correlation between the RIP volumes for the abdomen and thorax. As discussed above, anti-correlation (i.e., a correlation of negative one) occurs during paradox when the signals are out of phase, and during periods of normal respiration the correlation is positive. In FIG. 7C, the vertical axis is the ratio of the pneumotach flow to the RIP flow (each averaged over a short period). The whiter regions have higher concentrations of data having the corresponding x,y values for the RIP correlation and the ratio pneumotach flow/RIP flow. By minimizing an error function (e.g., the RMS error) and assuming a predefined functional form, a curve fit can be obtained. Here, positive correlations generally correspond to the curve fit value of about one, and approach a value close to zero (e.g., a value of 0.1) as the RIP volumes become anti-correlated.

Using the above-determined curve fit, the locally averaged correlation between the RIP volumes can be calculated and input to the curve fit to calculate a multiplier (i.e., the overestimation correction factor (OCF)). This multiplier may then be multiplied by the RIP flow to mitigate the overestimation during paradox. FIG. 7D shows the RIP flow that has been scaled by the OCF. Like in FIG. 7B, FIG. 7B illustrates the scaled RIP flow as a dark grey line, and illustrates the pneumotach flow as a black line. Here, much better agreement is observed between the scaled RIP flow and the pneumotach flow than was observed in FIG. 7B between the unscaled RIP flow and the pneumotach flow.

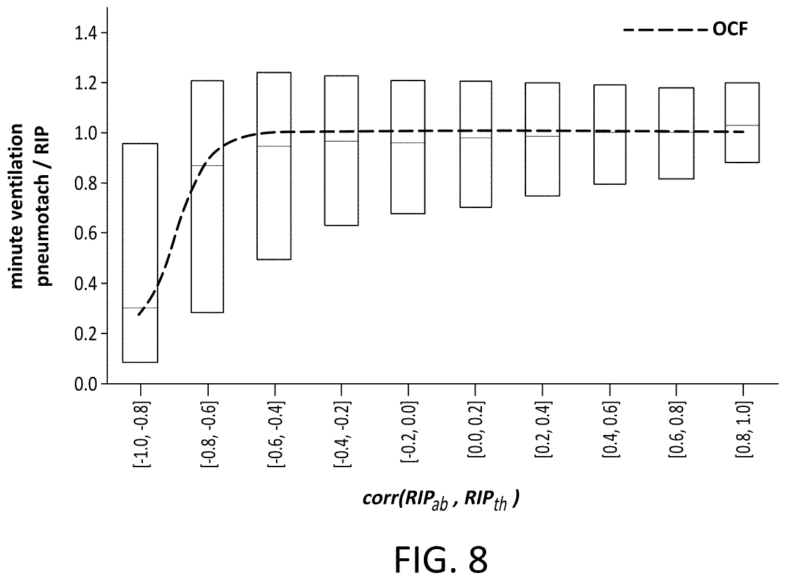

FIG. 8 illustrates another embodiment of determining the overestimation correction factor (OCF). To quantify the bias in the RIP flow during paradox and correct for it, the following analysis may be performed. First, the paradox is determined using the correlation coefficient R (Pearson) between thoracic and abdominal volume signals, for each breath. The oronasal ventilation and RIP ventilation are also calculated to examine whether the oronasal/RIP ventilation ratio was reduced with greater paradox. In FIG. 8 this relationship is plotted. More particularly, all breaths of data from multiple patients are sorted by R and binned into deciles. Next, median oronasal/RIP ventilation ratio are plotted against R. In this non-limiting embodiment, a sigmoid-shaped relationship was fit to the decile-binned data to describe the ‘overestimation correction factor (OCF)’. For this exemplary data set, the curve fit is

OCF=(1−a)[1/(e−20.92(R−0.0441)+1)2]+α and α=0.123.

Generally, OCF is approximately one except under severe paradox when it falls to as low as 0.12, for example. Multiplication of the calibrated RIP ventilation by OCF, on a breath-by-breath basis, may be used to obviate the systematic overestimation of ventilation during pharyngeal obstruction. Here, the OCF function is determined by using data from multiple patients, but in other embodiments, the particular values for the OCF function may be patient specific.

Returning to FIG. 1, a third calibration 126 is performed to determine and correct for RIP non-linearity. A plot of patient data with the RIP ventilation on one axis and pneumotach (e.g., oronasal) ventilation on the other axis may show a non-linear relation. For example, using RIP, ventilation may be underestimated for large breaths. Accordingly, a power-law correction factor may be applied to minimize this underestimation. In certain embodiments, the functional form of the power-law correction factor may be y=x1/β. For certain patients, it has been observed that when values of 0.75, 0.8, 0.85, 0.9 were attempted for the variable β, the optimal value was observed to be β=0.85.

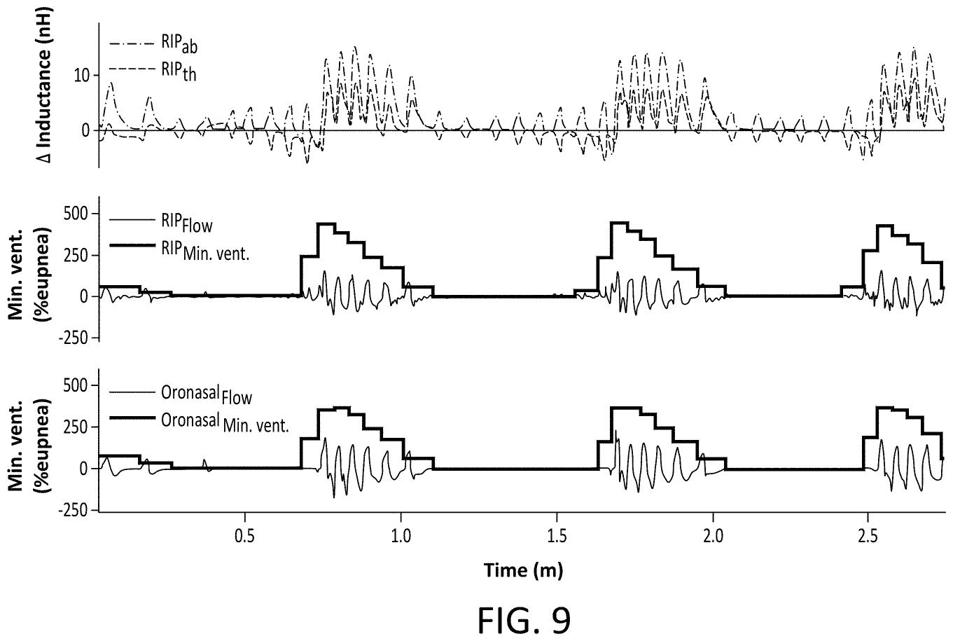

In FIG. 1, step 120 in which the calibrations are performed is followed by step 130 in which the calibrations are applied to the RIP data to generate a time series of RIP flow/ventilation values. In certain embodiments, the RIP flow/ventilation values are generated without measurements of the oral or nasal breathing. FIG. 9 illustrates starting with inductance measurements from the RIP belts, and converting these to flow values and minute ventilation values. The upper plot shows as a function of time (horizontal axis) the RIP inductance values for the abdomen and thorax. The middle plot shows the RIP flow signal derived from the time derivative of the calibrated RIP signal. Superimposed is the respective minute ventilation signal. The upper plot shows the target oronasal pneumotach signal and the target minute ventilation signal. Using the equation,

RIP_flow=kdRIP_Ab+(1−k)dRIP_Th

A flow estimate can be calculated from the RIP signals by using the time derivative of the calibrated RIP signal, where dRIP_Ab and dRIP_Th are the time derivatives of the abdominal and thoracic RIP signals, respectively and k is the calibration constant. In FIG. 9, the recorded ventilation traces of a 3-minute slice of a sleep study during a period of repeated obstructive respiratory events. In the upper plot, RIP inductance signals are shown with the Functional Residual Capacity (FRC) baseline removed to better visualize paradoxical movement during obstructive apnea periods.

Minute ventilation may be calculated by multiplying the tidal volume times the respiratory rate for breaths detected in wake and sleep. Values for the minute ventilation may be expressed as a percentage of the local average “eupneic” ventilation (7-min window). Here, “eupnea” means normal respiration, which contrast with the meaning “apea,” i.e., a temporary cessation of breathing, especially during sleep.

As discussed above, the flow and minute ventilation calculated from the RIP data are improved through novel RIP calibration methods with the aim of achieving an unbiased estimate of minute ventilation from the RIP signals, compared to the gold standard oronasal pneumotach, during spontaneous breathing in sleep. For example, the calibrated RIP data may be used to determine minute ventilation and OSA endotype values that agree closely with ventilation and OSA endotype values determined using the gold standard oronasal pneumotach, validating the RIP-based method for determining flow, ventilation, and OSA endotype values. Accordingly, the methods described herein provides clinicians with the tools to make more informed decisions when diagnosing and treating patients with OSA in a standard PSG study.

Returning to FIG. 1, in step 140, endotyping is determined from the flow and/or ventilation values determined from the RIP measurements. Endotyping is useful for diagnosing and treating sleep disorders. In light of increased skepticism about using the apnea-hypopnea index as the sole indicator of OSA, endotyping can provided needed insight for treating OSA. The methods described herein improve the accessibility of OSA endotyping by developing a RIP calibration method that allows for the extraction of endotypes from the RIP signals. The validity of these methods is demonstrated by the high agreement between RIP and pneumotach derived endotypes, for all explored OSA endotypes (upper airway collapsibility, upper airway muscle compensation, arousal threshold and loop gain).

For example, the determination of the underlying cause of sleep apnea in patients has been determined by measuring loop gain, determining the level of arousability of the patient, and by measuring the collapsibility of the upper airway. Traditionally, the loop gain is measured or estimated by measuring the change in respiratory drive as a response to change in ventilation, and the arousability is determined by measuring the respiratory drive resulting in a cortical arousal of a patient.

A phenotype is the composite of an organism's observable characteristics or traits, such as its morphology, development, biochemical or physiological properties, behavior, and products of behavior. A phenotype may be a result of the genetic expression in an organism, endophenotype. Phenotyping a subjects respiratory effort may reveal information about the mechanics of the respiratory system, the response of the system to a disturbance such as reduced ventilation, the tendency of the airway to collapse, the ability of dilator muscles in the system to maintain an open airway, the internal pressures and forces acting on parts of the respiratory system or in its entirety, the recruitment of respiratory muscles, and any other function resulting in breathing or caused by breathing.

An endotype is a subtype of a condition, which is defined by a distinct functional or pathobiological mechanism. This is distinct from a phenotype, which is any observable characteristic or trait of a disease, such as development, biochemical or physiological properties without any implication of a mechanism. It is envisaged that patients with a specific endotype present themselves within phenotypic clusters of diseases.

OSA endotyping is a method for identifying the pathophysiological traits that contribute to OSA. For example, these traits may include upper airway collapsibility, upper airway muscle compensation, arousal threshold and loop gain. Understanding the pathogenesis of OSA for individual patients can be used for targeted treatment. Endotyping may be an integral part of precision OSA diagnosis and treatment.

OSA endotypes are derived from patients' breathing patterns, which are generally captured with flow sensors. The main variable of interest is the amount of ventilation during each minute of sleep (minute ventilation), which may be the basis for endotype determination. Calculating endotypes is validated in laboratory settings using the gold standard pneumotach with a sealed oronasal mask. When used with an oronasal mask, the pneumotach measures unbiased flow and circumvents possible confounding effects of oral ventilation. The methods described here take the additional step to scaling the pneumotach method to RIP results, which are available in Polysomnographic (PSG) sleep studies such that endotyping can be applied to standard PSG recordings.

As discussed below with reference to an experimental study, when applying endotyping to standard PSG studies, it is important to validate its performance by comparing endotypes gathered from PSG flow sensors to endotypes from the gold standard oronasal pneumotach. The ideal comparison is within the same study by paired intra-patient comparisons.

When applying endotyping to standard PSG studies, it is necessary to validate its performance by comparing endotypes gathered from PSG flow sensors to endotypes from the gold standard oronasal pneumotach. The ideal comparison is within the same study by paired intra-patient comparisons. In a standard PSG sleep study, a nasal cannula is used for measuring nasal ventilation. The feasibility of using the nasal cannula to detect a subset of the OSA endotypes, namely: upper airway collapsibility (Vpassive) and upper airway muscle compensation (Vcomp) as well as ventilation at the arousal threshold (Vactive) [Sands et al. 2018], has been verified on a small cohort [PUPpy, Sands2018]. However, the nasal cannula may become dislodged during sleep, resulting in a lack of signal [Gu\check{o}nadóttir et al., 2019, Altman et al., 2012]. Further, validating this method is challenging due to the difficulties of simultaneous pneumotach and nasal pressure measurements and the possibility of one measurement influencing the other.

Even though the disclosure primarily discusses estimating endotypes, the methods described herein can use the RIP flow and/or RIP ventilation to estimate either phenotypes or endotypes.

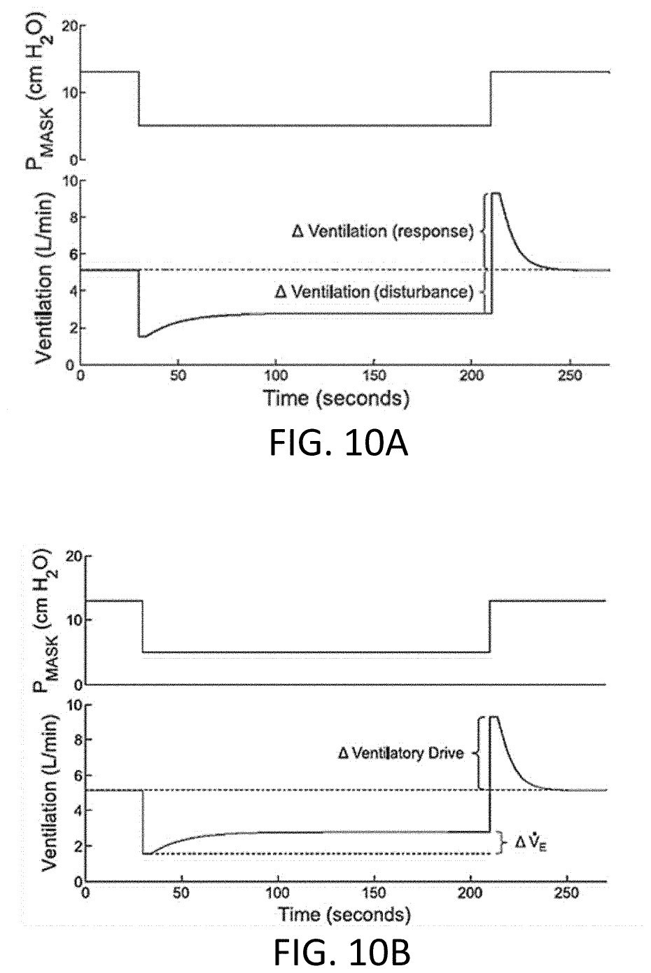

FIG. 10A illustrates a known Continuous Positive Airway Pressure (CPAP) maneuver and the resulting ventilatory response used in an endotyping protocol. The endotyping protocol involves having a patient on a CPAP at a therapeutic level. The CPAP level is lowered in an instant and kept at a lower than therapeutic pressure for a period of 3 minutes. The CPAP level is then set to the original therapeutic level. During this maneuver, the patient ventilation is measured. When the CPAP is at a therapeutic level the patient has unobstructed breaths, which are used as a reference for the later breaths. Once the CPAP is turned down ventilation is reduced. By increasing ventilatory drive, the patient manages to increase ventilation to a new steady state level. The difference between the ventilation during therapeutic CPAP and the ventilation at the new steady state is the ventilation disturbance. Once the CPAP is turned back on the airway opens up and the ventilation equals the ventilatory drive. The difference between the ventilation after turning the CPAP up and the normal unobstructed ventilation is the ventilatory response. The loop gain, LG, is found by the equation:

$$LG = \frac{D i s turbance}{Response}$$

The upper airway gain, UAG, is found by measuring how much the change in ventilatory drive changes ventilation as shown in FIG. 10B. FIG. 10B shows the ventilatory response to changes in CPAP pressure. The increase in ventilation due to increased ventilatory drive is labeled as Δ\dot{V}_{E}. The UAG is calculated by the equation:

$$U A G = \frac{Δ {\dot{V}}_{E}}{Δ Drive}$$

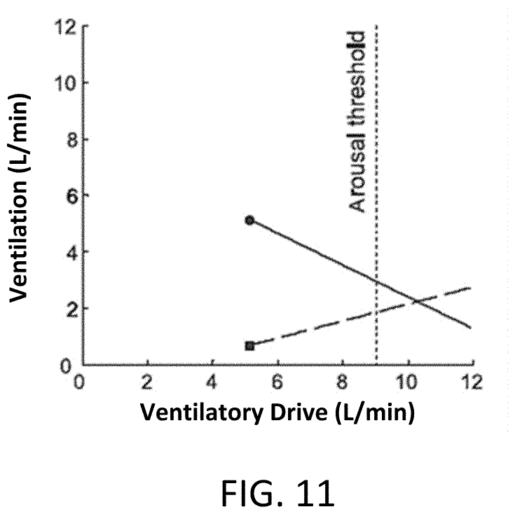

To be able to endotype a patient, data needs to be collected to build a similar figure as show in FIG. 11. FIG. 11 shows the eupneic ventilation, the ventilation during non-obstructed breathing. At this ventilation point the ventilation and ventilatory drive are equal and the breath can be thought of as a normal breath. The black line in the figure has a slope of 1/LG. LG is the loop gain, which is found by dividing the ventilation disturbance by the ventilation response.

In FIG. 11, data used in endotyping a patient with all four physiological traits plotted. The black circle indicates the eupneic ventilation, unobstructed breathing. The black line has a slope equal to 1/LG. The black square marker indicates the ventilation at zero mask pressure. The dashed line has a slope equal to UAG. The dotted line indicates the patient arousal threshold.

In FIG. 11, the black square marker is found by gradually dropping the CPAP pressure to lower levels and measuring the resulting ventilation. A line can be fit to this data and extrapolated to get the ventilation at a CPAP pressure of 0 cmH2O.

The arousal threshold is found by dropping the CPAP to a low-pressure level causing an arousal. The arousal threshold is calculated by fitting a model to the data and estimating the ventilatory drive at arousal.

Loop gain is a parameter in a model of the ventilatory control system which determines how ventilatory drive changes with respect to changes in arterial blood gases. The ventilatory drive is modeled as the sum of the chemical drive, the response to carbon dioxide, and a nonchemical drive accompanying an arousal

V_{drive}=V_{chem}+Vₐᵣₒᵤₛₐₗ

The chemical drive is described by a differential equation

$$τ \frac{{dV}_{chem}}{dt} = - {V}_{cham} - L {G}_{0} \times {V}_{E} ( t - δ )$$

τ is the characteristic time constant due to time course of the buffering of carbon dioxide in the lung and tissue, LG₀ is the steady state loop gain, and V_{E} is the ventilation. V_{E} (t−δ) is the previous level of ventilation where the delay δ is due to the time delay between the lung and chemoreceptors. Vₐᵣₒᵤₛₐₗ is a constant increase in ventilatory drive, γ, and accompanies scored EEG arousals.

A method has been introduced to calculate the loop gain, chemical drive, and response to an arousal from flow data and scored arousals. FIG. 12 shows an example of how the introduced algorithm performs. FIG. 12 shows artificial data where the ventilation, purple trace, was created by a model with known parameters. The purple areas indicate periods of an obstruction, and the green line with squares at the top are scored arousals. The black trace is the calculated chemical drive, and the green trace is the ventilatory drive which include the chemical drive plus the response to an arousal. FIG. 12 thus shows a modelled ventilation trace and scored arousals. The purple trace shows the measured ventilation normalized by the ventilation at a normal breath. The shaded areas indicate periods of obstruction, the green line is the calculated ventilatory drive, the black solid curve the calculated chemical drive.

A second method of identifying the loop gain is by identifying the eupneic flow or ventilation, F_{E}, during an unobstructed breath. During an airway obstruction the flow or ventilation is reduced. Immediately, the next breath, following an opening in the airway the flow or ventilation will increase dramatically, a recovery breath, Fₙ₊ᵢ=F_{E}+F_{recovery}, where i is a small number larger than 0 and typically smaller than 3. By calculating the ratio between the response F_{recovery}, and the disturbance ΔF found by the flow in a breath just preceding the change from an obstructed period to an unobstructed period Fₙ₋ⱼ=F_{E}−ΔF, where j is a small number larger than or equal to 0 and typically smaller than 5. The loop gain can be found by LG=F_{recovery}/ΔF.

Referring to FIG. 10A, a third method of identifying the loop gain is identifying the parameters of the model. During a normal breath the respiratory drive, Pₘᵤₛ, and upper airway resistance, R_{U}, are identified, and the eupneic ventilation, F_{E}, is estimated by calculating the current through the upper airway resistance. During an obstructed breath the drive, Pₘᵤₛ, and upper airway resistance, R_{U}, are identified and the obstructed ventilation, F_{obstructed}, is estimated by calculating the current through the upper airway resistance. The difference between the eupneic ventilation and the obstructed ventilation is the disturbance, F_{disturbance}=F_{E}−F_{obstructed}. By keeping the circuit elements with the values identified during the obstructed breath the value of the upper airway resistance can be changed to the value identified during eupneic breathing. By calculating the current through the upper airway resistance using the Pₘᵤₛ identified during the obstructed breathing and the R_{U} identified during the eupneic breathing the intended ventilation, F_{intended}=F_{E}+Fᵣₑₛₚₒₙₛₑ, can be calculated as the sum of the eupneic ventilation and the ventilation response. The loop gain can be found by LG=F_{disturbance}/Fᵣₑₛₚₒₙₛₑ.

The upper airway gain can be identified by comparing the changes in respiratory flow, respiratory drive Pₘᵤₛ, and upper airway resistance Rᵤ.

One method of identifying the upper airway gain is by identifying periods where breathing is unobstructed. During unobstructed breathing R_{U} is the same, or similar, during inhalation and exhalation; or the respiratory drive is linearly proportional to the flow; or the phases of all frequency components of i_{ab} are the same as the phases of all frequency components of iₜₕ. During the unobstructed periods a baseline drive, P_{mus E}, can be identified. At a nearby place in time, where ventilation is obstructed, the flow is reduced to a minimum, F₀. Following the reduction in flow the respiratory drive increases and becomes Pₙ=P_{mus E}+ΔPₘᵤₛ. The increase in respiratory drive causes an increase in flow from the minimum flow Fₙ=F₀+ΔF. The upper airway gain can be found by UAG=ΔF/ΔPₘᵤₛ.

The arousal threshold can be determined by recording the level of respiratory drive, Pₘᵤₛ, just before or at the instance of a recorded arousal.

By comparing the properties of the model to measurements with scored arousals it is possible to construct a metric of the probability of an arousal, Respiratory Arousal Probability (RAP). The RAP could be an indicator of the probability of a certain breath was followed by a cortical arousal, the probability of a certain breath occurring during a cortical arousal, or the probability of a breath following a cortical arousal. The RAP is determined by calculating the elements of the model, or looking at other properties of the RIP signals or flow signals, such as the signal entropy, the ratio of high frequency power to low frequency power, or the relative power in several power bands, and calculating the probability of a breath identified with a set of these parameters to occur before, during, or after an arousal. The method could include combining either the absolute or relative the properties of several consecutive breaths. Therefore, knowing the properties of one or many breaths can predict the probability of a cortical arousal occurring.

A different method of determining the RAP is to measure the time from a breath with certain parameters occurring to the next cortical arousal following the breath. Therefore, knowing the properties of one or many breaths can predict the length of time until next cortical arousal or predict if a cortical arousal will occur within a certain amount of time.

Additional methods for endotyping based on flow and or ventilation values are described in E. Finnsson et al., “A scalable method of determining physiological endotypes of sleep apnea from a polysomnographic sleep study,” Sleep, vol. 44, no. 1, p. zsaa168, January 2021, which is incorporated herein in its entirety.

Now, experimental data is presented illustrating the validity of the methods described herein. 71 patients with OSA underwent polysomnographic studies with RIP and oronasal ventilation. To estimate ventilation from RIP, algorithms were applied to handle thoracoabdominal calibration (e.g., the ‘powerloss’ method), overestimation during paradox, and non-linearity, as discussed above with reference to FIG. 1. Resulting values of RIP ventilation were compared against oronasal ventilation as a gold standard. It was subsequently examined whether RIP provided reliable measures of respiratory event depth and endotypic traits (loop gain, arousal threshold, collapsibility, compensation) via intra-class correlation (ICC) analysis.

The results of this analysis show that Respiratory inductance plethysmography can be used to reliably estimate ventilation and the traits causing OSA. More particularly, RIP calibration and linearization reduced the overestimation of ventilation during paradox (median bias: 7.8 to 0.9% eupnea) and the underestimation of ventilation during larger breaths (median bias: −20.3 to −6.9% eupnea). RIP ventilation measures of respiratory event depth (individual event: ICC=0.65 in 15927 events); patient average depth: ICC=0.85) and endotypic traits (ICC range=0.74-0.92) exhibited strong correlations with gold standard measures.

As discussed above, an airflow estimate can be derived from respiratory inductance plethysmography (RIP), which provides a surrogate measure of breathing through thoracic and abdominal movement. Like the nasal cannula, RIP is part of a standard PSG recording and is recommended by the American Academy of Sleep Medicine (AASM) for monitoring respiratory effort and as a secondary airflow sensor. With proper measurement and signal processing, RIP can be used to accurately and reliably measure ventilation independent of breathing route. Importantly, the pneumotach/mask does not interfere with RIP measurements, so simultaneous measurement requires no adjusted setup. Successful validation of endotyping from RIP could therefore make OSA endotyping easily available in the clinical setting. An integral step in this methodology is the calibration of the RIP signal.

The experimental discussed below includes a secondary analysis of a larger observational cohort study to investigate extreme phenotypes of OSA in four participant groups: (1) obese OSA patients, (2) non-obese OSA patients; (3) obese healthy controls, and (4) non-obese healthy controls. Adult male and female participants were recruited from two sites. Patients were excluded from the parent study if they had a sleep disorder diagnosis requiring treatment other than OSA, had periodic limb movements with arousals (>20/hr) or had received surgical or other treatment that affects the upper airway or the gastrointestinal tract. Pregnant patients and those who take sedatives, hypnotics, narcotics, muscle relaxants, or benzodiazepines more than once a week were also excluded.

Patients attended a single in-laboratory overnight polysomnographic sleep study which included 6-channel electroencephalogram, electrocardiogram, pulse oximetry, and thoracic and abdominal respiratory inductance plethysmography (RIP). RIP data are unfiltered (DC-coupled) and linearly related to chest and abdominal circumference. In place of the nasal cannula usually included in clinical polysomnography, quantitative oronasal ventilatory flow was measured by pneumotach via a sealed oronasal mask (Hans Rudolph, Shawnee, Kans., USA). Sleep stages were manually scored in accordance with AASM guidelines [ref]. Apneas were scored based on a >90% reduction in airflow (oronasal flow). Hypopneas were scored based on a >=30% reduction in airflow (oronasal flow) and >=4% oxygen desaturation. Arousals were scored based on AASM guidelines; arousal start and end times were carefully marked for physiological endotyping (for endotypes: additional arousals that occurred within 3-10 sec of a prior arousal were also scored).

First, an approach to estimating ventilation from RIP was developed and evaluated against oronasal ventilation. Second, the methods to estimate ventilation from RIP was employed to calculate the severity of respiratory events and to estimate the endotypic traits causing OSA (loop gain, arousal threshold, collapsibility, compensation).

Minute ventilation was calculated (tidal volume×respiratory rate) for breaths detected in wake and sleep; values were presented as a percentage of the local average “eupneic” ventilation (7-min window).

For each breath, ventilation was calculated from the RIP signals (‘RIP ventilation’) without any input from the pneumotach airflow reference signal.

The RIP signals were calibrated using the method described in FIG. 1. That is, three signal processing algorithms were sequentially applied to optimize the estimation of RIP ventilation: First, calibration involves finding the optimal ratio of abdominal to thoracic volume ‘k’ to sum to provide a surrogate tidal volume signal. Second, overestimation of tidal volumes under circumstances with complete paradox (e.g., during apnea, tidal volume should be minimal but uncorrected RIP can overestimate ventilation) was corrected for. Third, non-linearity in the form of underestimation of tidal volumes during larger breaths was corrected for.

RIP ventilation values were normalized for further analysis as a percentage of the local average “eupneic” RIP ventilation (7-min window).

The depth of each respiratory event (hypopneas and apneas; scored using oronasal ventilation) was calculated using RIP ventilation for comparison against event depth calculated separately from oronasal ventilation. Event depth was calculated for each manually scored respiratory event as the “nadir ventilation”; e.g., an event depth of 30% represents a 30% reduction in flow (from the moving-time mean ‘eupneic’ baseline level) and is the minimum flow-reduction requirement for a scored respiratory event (hypopnea). Adequate characterization of event depth was used for defining the clinically-scored events that define OSA. In addition, the average respiratory event depth for each subject was calculated; greater average event depth is a measure of greater upper airway collapsibility and a phenotypic biomarker of more severe OSA.

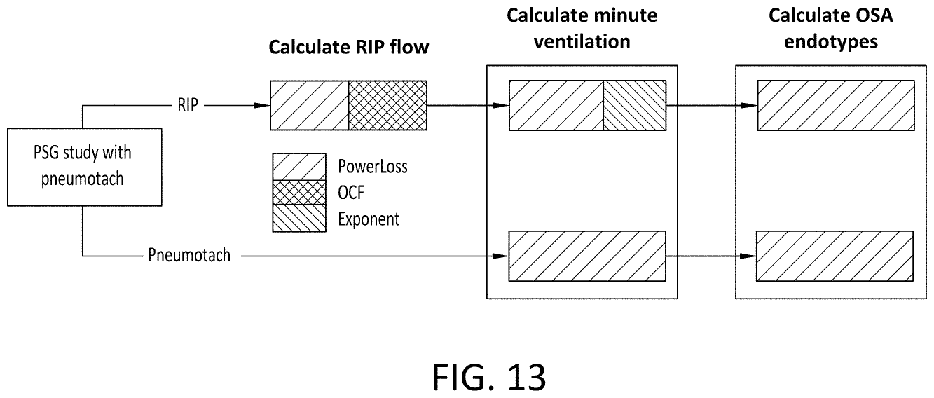

FIG. 13 illustrates a process for comparing/validating the RIP derived flow and endotypes against those derived via pneumotach. The OSA endotypes were calculated using RIP ventilation for comparison with separately-calculated traits using oronasal ventilation. OSA endotypes were calculated as described in E. Finnsson et al., “A scalable method of determining physiological endotypes of sleep apnea from a polysomnographic sleep study,” Sleep, vol. 44, no. 1, p. zsaa168, January 2021. Briefly, a physiological model was fit to the ventilation signal (for each 7-min window) between scored respiratory events to describe the ventilatory control system (gain, response time, delay), calculate the “loop gain” (value presented at 1 cycle/min), and provide an estimate of ventilatory drive. “Arousal threshold” was then calculated as the value of ventilatory drive preceding scored arousals. Median values of loop gain and the arousal threshold during eligible windows (at least 1 respiratory event, contains non-REM sleep, contains no REM sleep, contains no more than 30 s periods of wake) provided representative values for each patient. Collapsibility and compensation were calculated by first sorting all values of ventilatory drive during sleep (excluding breaths within arousals/wake, non-REM only) into multiple quantiles (N=20). Each quantile was characterized by median ventilatory drive and median ventilation values to provide a 20-point plot of ventilation vs. ventilatory drive, so called endogram. “Vpassive” was calculated as the ventilation at normal ventilatory drive (100% eupnea) and taken to represent collapsibility (lower values of ventilation reflect greater collapsibility). “Compensation” was calculated as the increase in ventilation (from Vpassive) when ventilatory drive rises to maximal levels during sleep (i.e., to the arousal threshold).

In the statistical analysis, regarding the RIP paradox overestimation, the “RIP paradox” was defined as breaths with −1.0<R<−0.8. The median bias for breaths in this is reduced after applying OCF. Regarding the RIP non-linearity, large breaths were defined as breaths where minute ventilation was >100% eupnea. Median large breath bias should be reduced after applying power law scaling. Regarding the event depth, the agreement between the event depth derived from 1) the calibrated RIP signals and 2) the oronasal pneumotachograph was evaluated using intraclass correlation (ICC). For individual breaths as well as per patient mean event depth. Regarding the normalization, arousal threshold and Vpassive values were projected onto a normal distribution using a square-root transform ArThres=1+(ArThres−1)0.5 and Vpassive=1−(1−Vpassive)0.5. Regarding the OSA endotypes, the agreement between the endotypes derived from 1) the calibrated RIP signals and 2) the oronasal pneumotachograph was investigated by using ICC, and Bland-Altman (BA) mean error and agreement. Intraclass correlation coefficient (ICC) was calculated using two-way mixed effects, with a single rater and absolute agreement. Confidence intervals were calculated using bootstrap, randomly sampling sleep studies with replacement for 10,000 iterations. The following table (Table 1) illustrates participant characteristics for the experimental study.

TABLE 1
Cohort information

| | Participants |
|---|---|
| | with OSA |
| N | 71 |
| Gender (M/F) | 58/13 |
| Ethnicity | |
| Caucasian | 57 |
| African-American | 10 |
| Asian | 3 |
| Other | 1 |
| AHI (mean/SD) [events/hour] | 37.38 (28.08) |
| Age (mean/SD) [yrs] | 53.35 (11.33) |
| BMI (mean/SD) [kg/m2] | 34.19 (6.66) |
| mean REM % duration (stdv) | 10.63 |
| mean N1 % duration (stdv) | 27.56 (18.76) |
| mean N2 % duration (stdv) | 50 (13.15) |
| mean N3 % duration (stdv) | 11.82(10.00) |
| mean time in supine in minutes | 55.53 (32.32) |
| (stdv) | |
| mean TST in minutes (stdv) | 337.93 (99.29) |
| fraction in hypopnea HR AHI | 0.58 (0.31) |
| (stdv) | |
| fraction in central apnea (stdv) | 0.03 (0.06) |
| fraction of events ending in | 0.39 (0.08) |
| arousal* | |

*An event was considered to end in arousal if an arousal occurred within 10 seconds of the end of the event

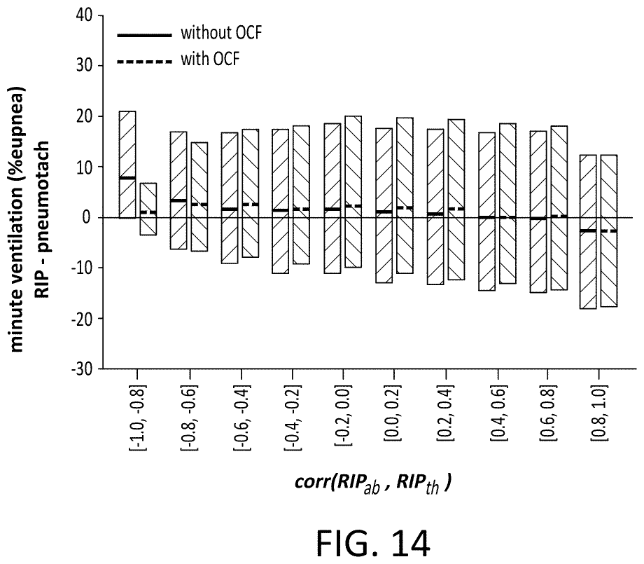

FIG. 14 illustrates experimental results for the RIP paradox overestimation for results with and without applying the OCF. More particularly, FIG. 14 shows the absolute minute ventilation error before and after applying OCF. As can be seen, the OCF reduces the median bias of RIP ventilation during periods of paradoxical breathing from 7.8% eupnea to 0.9% eupnea (FIG. 4). A total of 381.812 breaths were analyzed, 20.707 of which were in the (−1, −0.8) bin.

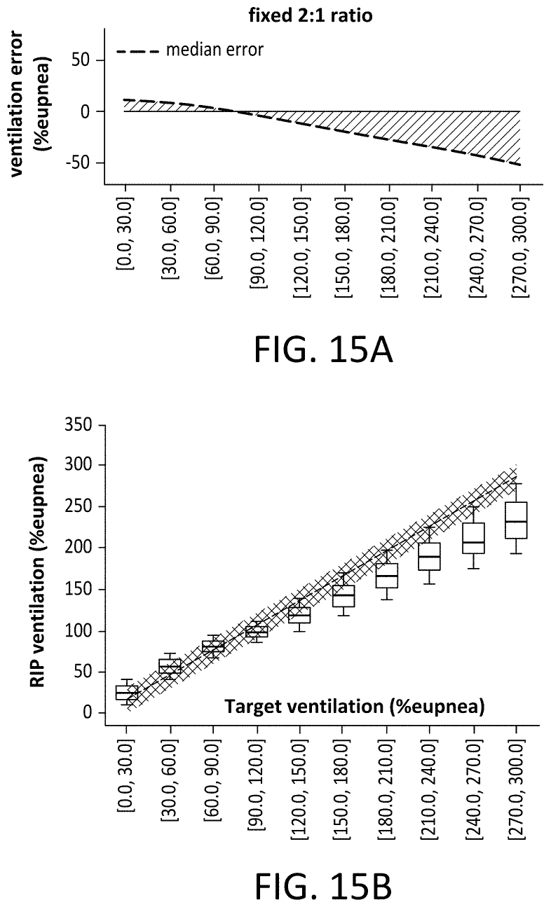

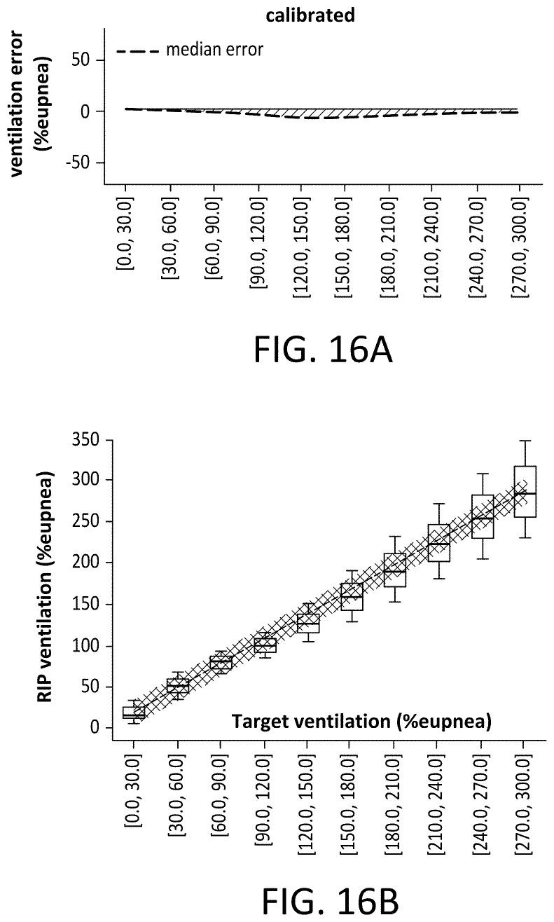

FIGS. 15A, 15B, 16A, and 16B illustrate experimental results related to the RIP non-linearity. FIGS. 15A and 15B show the results without the non-linearity correction, and FIGS. 16A and 16B show the results with the non-linearity correction. More particularly, these figures show the effects of calibration on different ranges of ventilation from small breaths to the left to large breaths to the right. In FIGS. 15B and 16B, the dashed line is the line of unity y=x and the shaded region shows the bin edges. In FIGS. 15A and 16A, the grey line shows the median error within each drive bin and the shaded region represents the median absolute error within each drive bin. As can be seen, the median large breath bias is reduced from: −20.3 to −6.9% eupnea. A large breath is defined as all breaths where ventilation >100% eupnea.

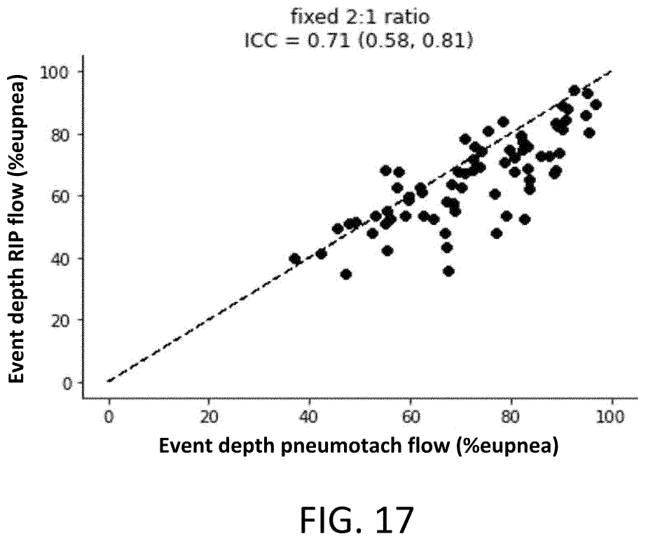

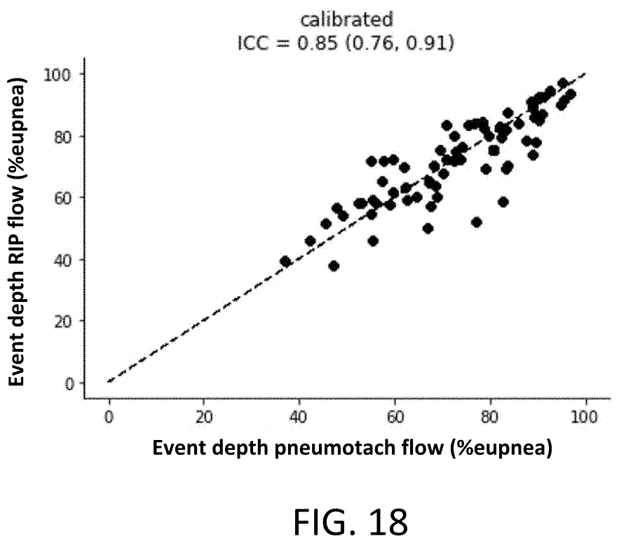

FIGS. 17 and 18 illustrate experimental results related to the event depth. FIG. 17 illustrates results for a fixed value of the calibration constant k, and FIG. 18 illustrates results when the calibration constant k is determined using the above-described methods. More particularly, these figures show the average event depth for n=71 sleep studies. The dashed line on the scatter plots is the line of identity (y=x). Each figure also contains the ICC of between the two methods of deriving average event depths with the 95% confidence interval. In FIG. 17, the calibration constant k, which is a fixed 2:1 ratio, resulted in an ICC value of 0.71 (0.58, 0.81), median (95p CI). In FIG. 18, the calibrated calibration constant k resulted in an ICC value of 0.85 (0.76, 0.91).

Table 2 summarize result for the OSA endotypes. The calibrated RIP derived OSA endotype values are compared to the values derived from the gold standard oronasal pneumotach. Table 2 shows the summary statistics for each endotype. For all endotypes, there is high agreement between the two derivation methods.

TABLE 2
Summary statistics describing the agreement between RIP
endotypes after POE calibration of the RIP signals and pneumotach
endotypes: Intraclass Correlation Coefficient (ICC), Pearson
correlation coefficient (PCC), Bland-Altman bias and limits of
agreement (Pneumotach-RIP) and mean absolute error as well as
the mean absolute error.

| | | | Mean | 95% agr. | mean |
|---|---|---|---|---|---|
| | ICC | PCC | error | interv. | abs error |
| ArThres | 0.92 | 0.92 | −1.29 | 23.24 | 9.40 |
| | (0.87, 0.95) | (. . ., . . .) | (. . ., . . .) | (. . ., . . .) | (. . ., . . .) |
| Vactive | 0.88 | 0.89 | −5.40 | 32.30 | 12.25 |
| | (. . ., . . .) | (. . ., . . .) | (. . ., . . .) | (. . ., . . .) | (. . ., . . .) |
| Vpassive | 0.92 | 0.92 | 1.45 | 18.61 | 7.04 |
| | (. . ., . . .) | (. . ., . . .) | (. . ., . . .) | (. . ., . . .) | (. . ., . . .) |
| Vcomp | 0.74 | 0.76 | −5.20 | 31.03 | 11.95 |
| | (. . ., . . .) | (. . ., . . .) | (. . ., . . .) | (. . ., . . .) | (. . ., . . .) |
| LG1 | 0.78 | 0.89 | 0.06 | 0.27 | 0.12 |
| | (. . ., . . .) | (. . ., . . .) | (. . ., . . .) | (. . ., . . .) | (. . ., . . .) |

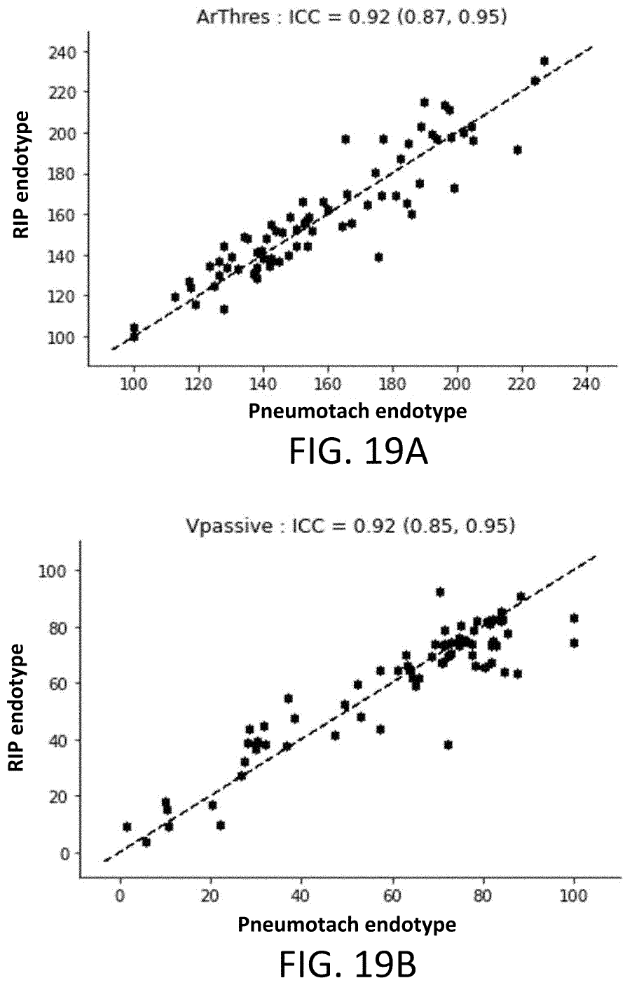

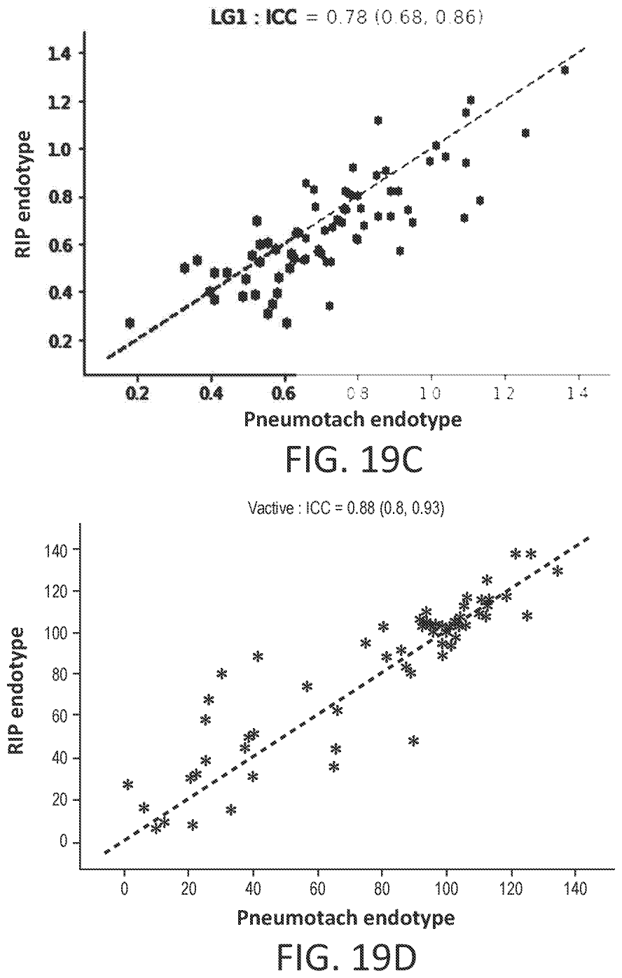

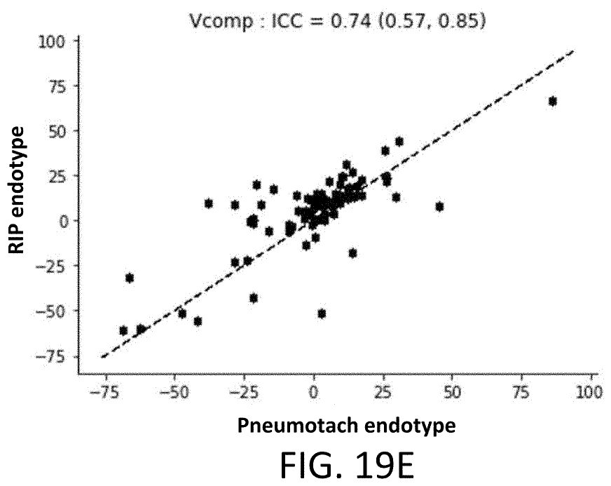

FIGS. 19A-19E 5 show the ICC values for the comparison of the endotypes derived using RIP measurements versus endotypes derived using pneumotach measurements. The ICC values for the endotypes were found to be 0.92 for arousal threshold, 0.78 for LG1, 0.92 for Vpassive, 0.88 for Vactive, and 0.74 for Vcomp. More particularly, these figures compare the endotypes as derived from the two methods of measurement RIP vs. the gold standard oronasal pneumotach. Each datapoint represents a full sleep study of a single participant. The dashed line on the scatter plots is the line of identity (y=x). Each figure also contains the ICC of the respective endotype with the corresponding 95% confidence interval.

FIG. 19A shows the comparison for the arousal threshold endotype. FIG. 19B shows the comparison for the upper airway collapsibility endotype. FIG. 19C shows the comparison for the loop gain endotype. FIG. 19D shows the comparison for the ventilation at the arousal threshold endotype. FIG. 19D shows the comparison for the upper airway muscle compensation endotype.

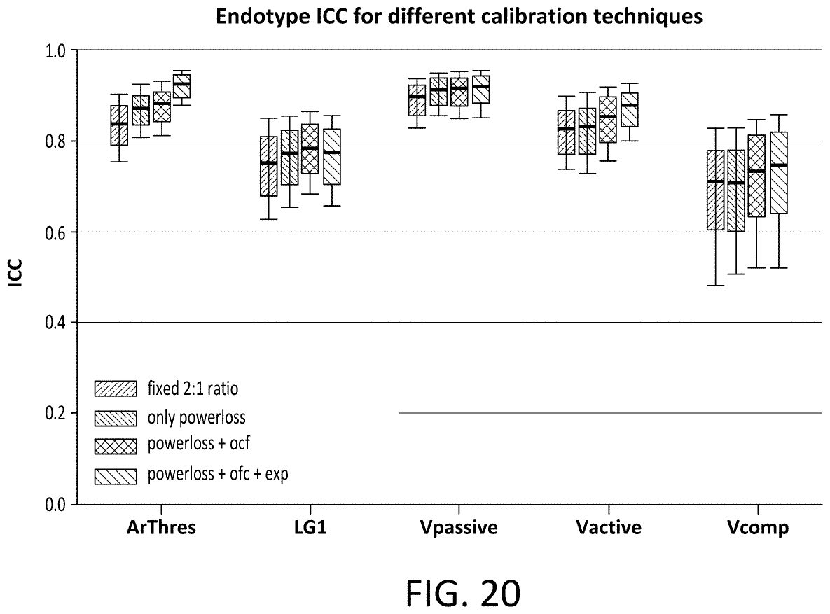

FIG. 20 shows ICC between RIP derived endotypes and the gold standard pneumotach derived endotypes for different subsets of the RIP calibration technique. Confidence intervals were calculated using bootstrapping. The ICC was calculated for each endotype using different levels of calibration on the RIP signals. FIG. 6 shows boxplots of the ICC values when the RIP is calibrated using the 1:2 ratio, PowerLoss, PowerLoss and OCF, and PowerLoss, OCF and exponential scaling. The figure shows how the calibration methods impact the ICC.

As can be seen in the experimental results, OSA endotypes can be successfully derived from PSG sleep studies using RIP belts to measure minute ventilation. Further, using the methods described herein, the limitations of linear RIP calibration methods can be supplemented using nonlinear calibration methods.

The methods described herein improve the accessibility of OSA endotyping by developing a RIP calibration method that allows for the extraction of endotypes from the RIP signals. The above experimental results demonstrate this, as shown by the high agreement between RIP and pneumotach derived endotypes, for all explored OSA endotypes (upper airway collapsibility, upper airway muscle compensation, arousal threshold and loop gain).

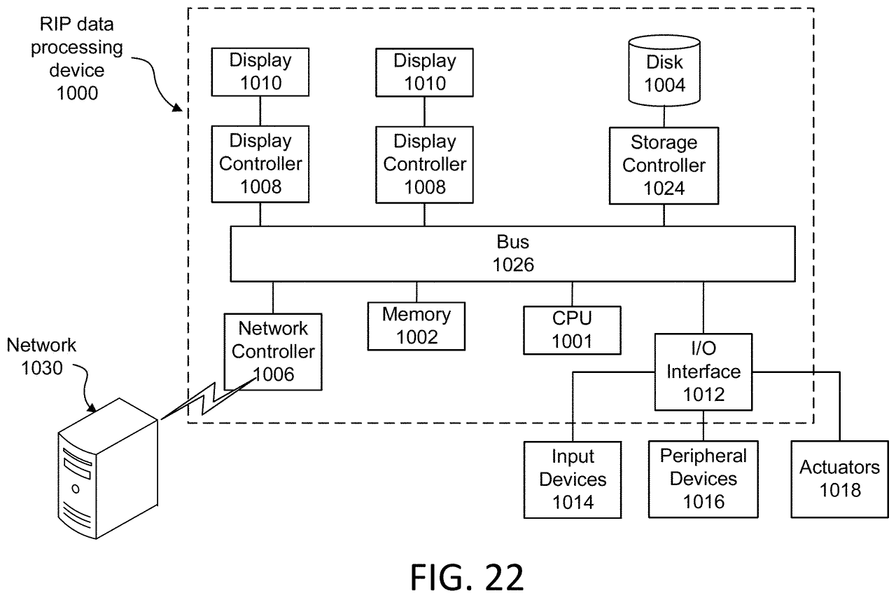

FIG. 22 illustrates a device 1000 configured to perform method 100 and process the RIP data to generate flow values, ventilation values, and/or endotypes. The device 1000 can perform some or all of the steps discussed above. The device 1000 may perform method 100 using a utility application, background daemon, or component of an operating system, or combination thereof, executing in conjunction with CPU 1001 and an operating system such as Microsoft Windows 7, UNIX, Solaris, LINUX, Apple MAC-OS and other systems known to those skilled in the art.

CPU 1001 may be a Xenon or Core processor from Intel of America or an Opteron processor from AMD of America, or may be other processor types that would be recognized by one of ordinary skill in the art. Alternatively, the CPU 1001 may be implemented on an FPGA, ASIC, PLD or using discrete logic circuits, as one of ordinary skill in the art would recognize. Further, CPU 1001 may be implemented as multiple processors cooperatively working in parallel to perform the instructions of the inventive processes described above.

The device 1000 in FIG. 22 also includes a network controller 1006, such as an Intel Ethernet PRO network interface card from Intel Corporation of America, for interfacing with a network 1030. The network 1030 can be a public network, such as the Internet, or a private network such as an LAN or WAN network, or any combination thereof and can also include PSTN or ISDN sub-networks. The network 1030 can also be wired, such as an Ethernet network, or can be wireless such as a cellular network including EDGE, 3G and 4G wireless cellular systems. The network 1030 can also be Wi-Fi, Bluetooth, or any other wireless form of a communication that is known.

The device 1000 further includes a display controller 1008 for interfacing with a display 1010. A general purpose I/O interface 1012 interfaces with input devices 1014 as well as peripheral devices 1016. The general purpose I/O interface also can connect to a variety of actuators 1018. The input devices 1014 can include the various sensors shown in FIGS. 3 and 4, for example. The input devices 1014 may include an interface to receive data from the signal processor 350 in FIG. 3, for example.

A sound controller 1020 may also be provided in the device 1000 to interface with speakers/microphone 1022 thereby providing sounds and/or music.

A general purpose storage controller 1024 connects the storage medium disk 1004 with a communication bus 1026, which may be an ISA, EISA, VESA, PCI, or similar, for interconnecting all of the components of the device 1000. Descriptions of general features and functionality of the display 1010, input devices 1014 (e.g., a keyboard and/or mouse), as well as the display controller 1008, storage controller 1024, network controller 1006, sound controller 1020, and general purpose I/O interface 1012 are omitted herein for brevity as these features are known.

Method 100 can be stored on computer storage media and performed using a computation/logic circuitry. For example, method 100 may be performed on a central processing unit (CPU). Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the disclosure.

Transmission media can include a network and/or data links which can be used to carry program code in the form of computer-executable instructions or data structures, and which can be accessed by a general-purpose or special-purpose computer system. A “network” may be defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions may comprise, for example, instructions and data which, when executed by one or more processors, cause a general-purpose computer system, special-purpose computer system, or special-purpose processing device to perform a certain function or group of functions. Computer-executable instructions may be, for example, binaries, intermediate format instructions such as assembly language, or even source code.

The disclosure of the present application may be practiced in network computing environments with many types of computer system configurations, including, but not limited to, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

The disclosure of the present application may also be practiced in a cloud-computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Some embodiments, such as a cloud-computing environment, may comprise a system that includes one or more hosts that are each capable of running one or more virtual machines. During operation, virtual machines emulate an operational computing system, supporting an operating system and perhaps one or more other applications as well. In some embodiments, each host includes a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

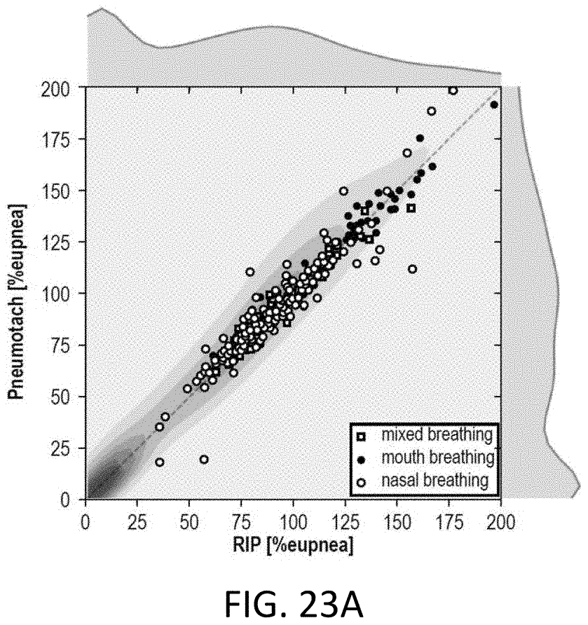

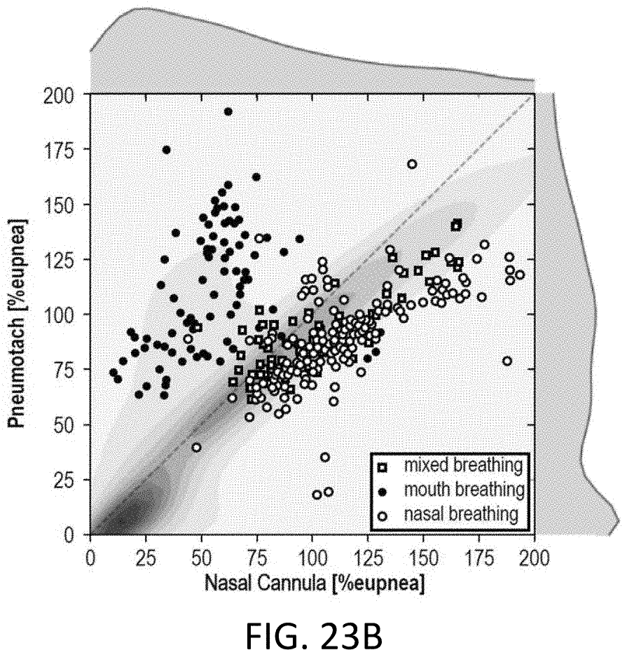

FIG. 23A illustrates a comparison in the determined event depths for three sets of data corresponding to mouth breathing, nasal breathing, and mixed breathing. The vertical axis represents the event depth determined based on pneumotach measurements. The horizontal axis represents the event depth determined based on RIP measurements. Close agreement between the approaches to determining the event depth is shown by the clustering of these results near the x=y line. Because pneumotach is taken as the gold standard, this close agreement indicates that RIP measurements provide an accurate approach to determine event depth.

FIG. 23B illustrates a comparison in the determined event depths for three sets of data corresponding to mouth breathing, nasal breathing, and mixed breathing. The vertical axis represents the event depth determined based on pneumotach measurements. The horizontal axis represents the event depth determined based on nasal cannula measurements. As might be expected, the nasal-cannula-based ventilation underestimate the ventilation for the mouth breathing data points. Thus, the RIP-based ventilation results appear to be better for predicting ventilation.

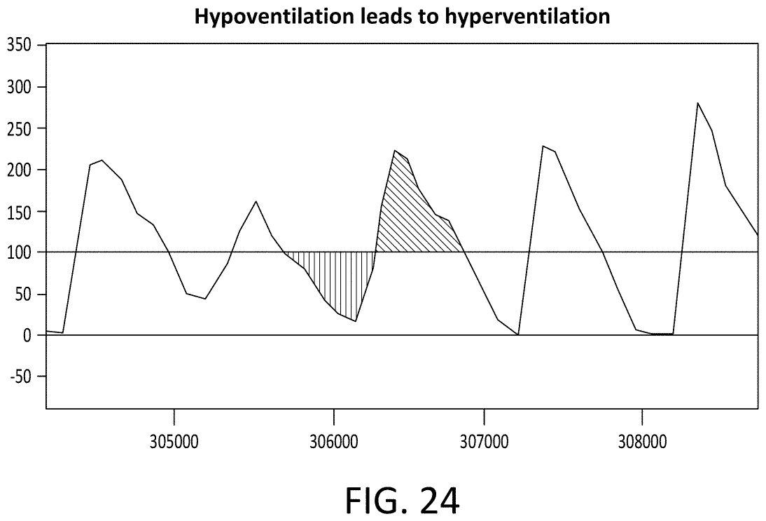

FIG. 24 illustrates ventilation with respect to time. A cycle of hypoventilation (indicated by the vertical stripes) followed by hyperventilation (indicated by the diagonal stripes) is shown. Hyperventilation allows recovery from the oxygen deprivation occurring during hypoventilation.

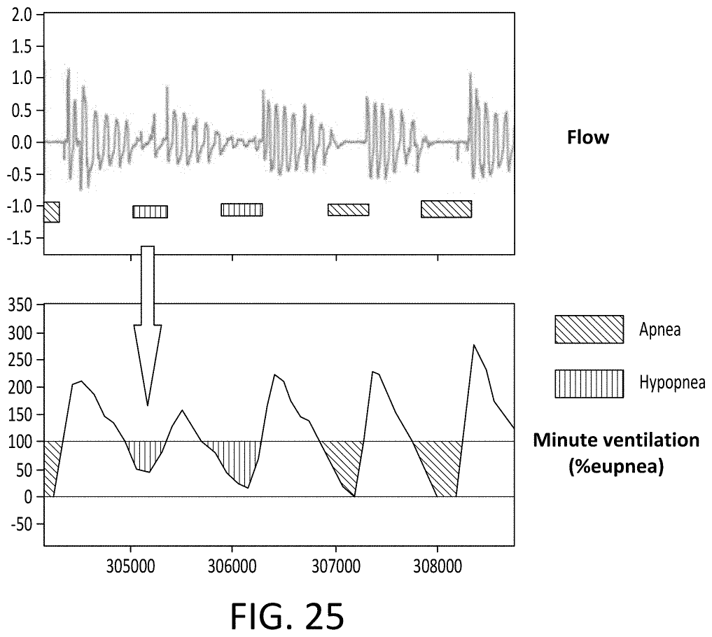

FIG. 25 illustrates flow and ventilation with respect to time. More particularly, FIG. 25 illustrates periods of apnea and hypopnea. Apnea is an OSA in which there occurs a full obstruction of the airway when a person is asleep. Hypopnea is an OSA in which there occurs a partial blockage of the airway.

Certain terms are used throughout the description and claims to refer to particular methods, features, or components. As those having ordinary skill in the art will appreciate, different persons may refer to the same methods, features, or components by different names. This disclosure does not intend to distinguish between methods, features, or components that differ in name but not function. The figures are not necessarily drawn to scale. Certain features and components herein may be shown in exaggerated scale or in somewhat schematic form and some details of conventional elements may not be shown or described in interest of clarity and conciseness.

Although various example embodiments have been described in detail herein, those skilled in the art will readily appreciate in view of the present disclosure that many modifications are possible in the example embodiments without materially departing from the concepts of present disclosure. Accordingly, any such modifications are intended to be included in the scope of this disclosure. Likewise, while the disclosure herein contains many specifics, these specifics should not be construed as limiting the scope of the disclosure or of any of the appended claims, but merely as providing information pertinent to one or more specific embodiments that may fall within the scope of the disclosure and the appended claims. Any described features from the various embodiments disclosed may be employed in combination. In addition, other embodiments of the present disclosure may also be devised which lie within the scopes of the disclosure and the appended claims. Each addition, deletion, and modification to the embodiments that falls within the meaning and scope of the claims is to be embraced by the claims.

Certain embodiments and features may have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges may appear in one or more claims below. Any numerical value is “about” or “approximately” the indicated value, and takes into account experimental error and variations that would be expected by a person having ordinary skill in the art.

This disclosure provides various examples, embodiments, and features which, unless expressly stated or which would be mutually exclusive, should be understood to be combinable with other examples, embodiments, or features described herein.

In addition to the above, further embodiments and examples include the following:

1. A method for determining a respiratory flow from respiratory inductance plethysmography (RIP) signals, the method comprising:

receiving data of a thoracic signal that corresponds to a length of a first RIP belt arranged proximate with a thorax of a subject;
receiving data of an abdomen signal that corresponds to a length of a second RIP belt arranged proximate with an abdomen of the subject; and
determining a respiratory flow based on the thoracic signal and the abdomen signal, wherein the respiratory flow is determined using two or more calibrations, including:
a first calibration applying a first calibration coefficient that relates an amplitude of a differential change in the thoracic signal to an amplitude of a differential change in the abdomen signal, and a second calibration that corrects for a non-linearity in the determined respiratory flow.

2. The method according to any or a combination of 1 above and/or 3-76 below, further comprising a step of determining one or more endotypes of an obstructive sleep apnea or of another sleep disorder of the subject based on the respiratory flow.

3. The method according to any or a combination of 1-2 above and/or 4-76 below, wherein determining the respiratory flow further includes a third calibration that corrects for an overestimation of flow during paradox.

4. The method according to any or a combination of 1-3 above and/or 5-76 below, wherein the thoracic signal represents a circumference of the thorax of the subject at a series of times, and the abdomen signal represents a circumference of the abdomen of the subject at the series of times.

5. The method according to any or a combination of 1-4 above and/or 6-76 below, wherein the thoracic signal and the abdomen signal respectively represent an inductance of the first RIP belt and the second RIP belt, and the method further comprises taking respective derivatives of the thoracic signal and the abdomen signal and combining the derivatives using a scaling coefficient to determine the respiratory flow.

6. The method according to any or a combination of 1-5 above and/or 7-76 below, wherein the determining of the respiratory flow includes

calculating a change of the thoracic signal with respect to time to generate a derivative corresponding to a time derivative of a thoracic volume,
calculating a change of the abdomen signal with respect to time to generate another derivative corresponding to a time derivative of an abdomen volume, and
the respiratory flow is determined by combining the derivative and the another derivative using a weighted sum that is based on the first calibration coefficient.
determining one or more endotypes of an obstructive sleep apnea or of another sleep disorder of the subject based on the respiratory flow.

7. The method according to any or a combination of 1-6 above and/or 8-76 below, wherein the determining of the respiratory flow includes calculating a time derivative of a calibrated sum of the thoracic signal and the abdomen signal, the calibrated sum being based on the first calibration coefficient.

8. The method according to any or a combination of 1-7 above and/or 9-76 below, wherein the first calibration is determined using the thoracic signal and the abdomen signal in an absence of all flow signals.

9. The method according to any or a combination of 1-8 above and/or 10-76 below, wherein the first calibration is determined using the thoracic signal and the abdomen signal in an absence of flow signals measured by one or more nasal canula.

10. The method according to any or a combination of 1-9 above and/or 11-76 below, wherein

the first calibration is carried out by selecting a value of the calibration coefficient that minimizes a function that represents a ratio of numerator to a denominator,
the numerator being a power of a weighted/scaled sum of the thoracic signal and the abdomen signal, wherein a weighting/scaling of the weighted/scaled sum is based on the value of the calibration coefficient,
the denominator being a power of the scaled thoracic signal summed with a power of a weighted/scaled abdomen signal, which is the abdomen signal that has been weighted/scaled sum is based on the value of the calibration coefficient, wherein
a weighting/scaling factor used to weight/scale the thoracic signal relative to the abdomen signal is the calibration coefficient or a function of the calibration coefficient.

11. The method according to any or a combination of 1-10 above and/or 12-76 below, wherein the first calibration is carried out by finding the value of k that satisfies to a predetermined threshold the equation

$${min}_{k} \frac{R M S ( ( 1 - k ) {dRIP}_{t h} + {kdRIP}_{a b} )}{R M S ( ( 1 - k ) {dRIP}_{th} ) + R M S ( {kdRIP}_{a b} )} ,$$

wherein RMS is the root mean square, k is a test value of the calibration coefficient, dRIPₜₕ represents a derivative of the thoracic signal with respect to time, dRIP_{ab} represents a derivative of the abdomen signal with respect to time.

12. The method according to any or a combination of 1-11 above and/or 13-76 below, wherein the first calibration is carried out using a PowerLoss calibration method.

13. The method according to 3 above or any or a combination of 1-12 above and/or 14-76 below, wherein the third calibration includes steps of

determining a correlation factor between the thoracic signal and the abdomen signal,
calculating an overestimation correction factor based on the correlation factor, and
the determining of the respiratory flow further includes scaling a derivative of a weighted sum of the thoracic signal and the abdomen signal by the overestimation correction factor.

14. The method according to 3 above or any or a combination of 1-13 above and/or 15-76 below, wherein the third calibration includes steps of

determining a correlation factor between the thoracic signal and the abdomen signal,
calculating an overestimation correction factor based on the correlation factor, and
the determining of the respiratory flow further includes scaling the thoracic signal and the abdomen signal by the overestimation correction factor.

15. The method according to any or a combination of 1-14 above and/or 16-76 below, wherein the second calibration includes steps of

calculating a non-linearity correction factor based on a curve fit, the curve fit having been generated from calibration data that includes the respiratory flow, which is uncorrected for the non-linearity, and a reference flow, which is measured using trusted/validated method and is acquired concurrently with the respiratory flow of the calibration data, and
the determining of the respiratory flow further includes scaling a derivative of a weighted sum of the thoracic signal and the abdomen signal by the non-linearity correction factor.

16. The method according to any or a combination of 1-15 above and/or 17-76 below, wherein the second calibration includes steps of

calculating a non-linearity correction factor based on a curve fit, the curve fit having been generated from calibration data that includes the respiratory flow, which is uncorrected for the non-linearity, and a reference flow, which is measured using trusted/validated method and is acquired concurrently with the respiratory flow of the calibration data, and
the determining of the respiratory flow further includes scaling the thoracic signal and the abdomen signal by the non-linearity correction factor.

17. The method according to 13 above or any or a combination of 1-16 above and/or 18-76 below, wherein the curve fit for the calculating of the non-linearity correction factor has an exponential functional form and the non-linearity correction factor is an exponent.

18. The method according to 14 above or any or a combination of 1-17 above and/or 19-76 below, wherein the curve fit for the calculating of the non-linearity correction factor has an exponential functional form and the non-linearity correction factor is an exponent.

19. The method according to any or a combination of 1-18 above and/or 20-76 below, wherein the determining of the respiratory flow is performed by first applying the first calibration and then applying the second calibration.

20. The method according to any or a combination of 1-19 above and/or 21-76 below, wherein the determining of the respiratory flow is performed by first applying the second calibration and then applying the first calibration.

21. The method according to 3 above or any or a combination of 1-20 above and/or 22-76 below, wherein the determining of the respiratory flow is performed by first applying the second calibration and then applying the third calibration.

22. The method according to 3 above or any or a combination of 1-21 above and/or 23-76 below, wherein the determining of the respiratory flow is performed by first applying the third calibration and then applying the second calibration.

23. The method according to 3 above or any or a combination of 1-22 above and/or 24-76 below, wherein the determining of the respiratory flow is performed by first applying the second calibration, then applying the third calibration, and then applying the first calibration.

24. The method according to 3 above or any or a combination of 1-23 above and/or 25-76 below, wherein the determining of the respiratory flow is performed by first applying the second calibration, then applying the first calibration, and then applying the third calibration.

25. The method according to 3 above or any or a combination of 1-24 above and/or 26-76 below, wherein the determining of the respiratory flow is performed by first applying the third calibration, then applying the second calibration, and then applying the first calibration.

26. The method according to 3 above or any or a combination of 1-25 above and/or 27-76 below, wherein the determining of the respiratory flow is performed by first applying the first calibration, then applying the second calibration, and then applying the third calibration.

27. The method according to 3 above or any or a combination of 1-26 above and/or 28-76 below, wherein the determining of the respiratory flow is performed by first applying the first calibration, then applying the third calibration, and then applying the second calibration.

28. The method according to any or a combination of 1-27 above and/or 29-76 below, further comprising steps of

determining periods of constant calibration by detecting changes a first DC signal, which is DC offset component of the thoracic signal, and/or a second DC signal, which is DC offset component of the abdomen signal, and indicating that periods between the detected changes are respectively periods of constant calibration, and
recalibrating the first calibration within each of the periods of constant calibration based on the thoracic signal and the abdomen signal within the respective periods of constant calibration.

29. The method according to 28 above or any or a combination of 1-28 above and/or 30-76 below, wherein, during a given period of the periods of constant calibration, the recalibrating of the first calibration includes obtaining a value of the first calibration coefficient that minimizes a ventilation that would be obtained from the thoracic signal and the abdomen signal during the paradox within the given period.

30. The method according to any or a combination of 1-29 above and/or 31-76 below, wherein the determining of the respiratory flow is performed without relying on direct measurement of oral breathing.

31. The method according to any or a combination of 1-30 above and/or 32-76 below, wherein the determining of the respiratory flow is performed without relying on direct measurement of nasal breathing.

32. The method according to any or a combination of 1-31 above and/or 33-76 below, wherein the determining of the respiratory flow is performed without relying on direct measurement of oral breathing and without relying on direct measurement of nasal breathing.

33. The method according to any or a combination of 1-32 above and/or 34-76 below, further comprising a step of determining ventilation based on the thoracic signal and the abdomen signal.

34. The method according to any or a combination of 1-33 above and/or 35-76 below, further comprising a step of determining ventilation based on the thoracic signal and the determined respiratory flow.

35. The method according to 2 above or any or a combination of 1-34 above and/or 36-76 below, wherein the step of determining the one or more endotypes further includes determining an endotype of upper airway collapsibility (Vpassive), upper airway muscle compensation (Vcomp), ventilation at the arousal threshold (Vactive), arousal threshold, and loop gain.

36. The method according to 2 above or any or a combination of 1-35 above and/or 37-76 below, further comprising a step of treating the subject based on the determined one or more endotypes.

37. The method according to 3 above or any or a combination of 1-36 above and/or 38-76 below, wherein the second calibration includes a curve fit that is personalized to the subject.

38. The method of any or a combination of 1-37 above and/or 39-76 below, further comprising:

determining ventilation based on the determined respiratory flow; and
quantifying relative changes in the determined ventilation and therefrom and detecting one or more respiratory events.

39. The method according to 38 above or any or a combination of 1-38 above and/or 40-76 below, wherein the one or more respiratory events include apneas, hypopneas, flow restriction, flow limitation, snoring, obstructed breathing, normal breathing, or eupnea.

40. The method according to 38 above or any or a combination of 1-39 above and/or 41-76 below, further comprising localizing the one or more respiratory events with respect to time.

41. The method according to 40 above or any or a combination of 1-40 above and/or 42-76 below, wherein the one or more respiratory events is hypoxic burden.

42. The method according to 40 above or any or a combination of 1-41 above and/or 43-76 below, wherein the localizing of the one or more respiratory events is used to improve accuracy of manual event scoring.

43. The method according to 40 above or any or a combination of 1-42 above and/or 44-76 below, wherein the localizing of the one or more respiratory events is used to improve event duration estimates.

44. The method according to 40 above or any or a combination of 1-43 above and/or 45-76 below, wherein the localizing of the one or more respiratory events further includes determining two points in time, a first point in time being a start time of a respiratory event and a second point being a stop time of the respiratory event, and the first point being determined by a predefined reduction in ventilation and the second point being determined by a recovery of ventilation.

45. The method according to 38 above or any or a combination of 1-44 above and/or 46-76 below, further comprising determining an event severity for the one or more respiratory events.

46. The method according to 45 above or any or a combination of 1-45 above and/or 47-76 below, wherein the event severity is a measure of upper airway patency and/or collapsibility.

47. The method according to 45 above or any or a combination of 1-46 above and/or 48-76 below, further comprising classifying respiratory events into apnea or hypopnea based on the event severity.

48. The method according to 45 above or any or a combination of 1-47 above and/or 49-76 below, wherein the event severity is determined by an event duration as measured by a time difference between a first point in time and a second point in time, the first point in time being a start time of a respiratory event and the second point being a stop time of the respiratory event, and the first point being determined by a predefined reduction in ventilation and the second point being determined by a recovery of ventilation.

49. The method according to 45 above or any or a combination of 1-48 above and/or 50-76 below, wherein the event severity is determined by and event depth, the event depth being a relative reduction in ventilation.

50. The method according to 45 above or any or a combination of 1-49 above and/or 51-76 below, wherein the event severity is determined by an apnea burden, the apnea burden being the cumulative loss of ventilation during the event.

51. The method according to 50 above or any or a combination of 1-50 above and/or 52-76 below, wherein the cumulative loss of ventilation is calculated by one of

integrating a reduction in ventilation for the event,
summing a difference between a baseline ventilation and a reduced ventilation during the event, determining a baseline ventilation as the ventilation at normal breathing, or
determining the baseline ventilation as the mean ventilation in a time window where the time window is long enough to require the person to maintain ventilation to sustain metabolism.

52. The method according to 38 above or any or a combination of 1-51 above and/or 53-76 below, wherein the one or more respiratory events is a recovery breath, and the method further comprises localizing the recovery breath with respect to time.

53. The method according to 52 above or any or a combination of 1-52 above and/or 54-76 below, wherein the recovery breath detection and localization are used to detect accurate respiratory arousal.

54. The method according to 52 above or any or a combination of 1-53 above and/or 55-76 below, wherein the recovery breath detection and localization are used to determine a first breath after the one or more respiratory events.

55. The method according to 52 above or any or a combination of 1-54 above and/or 56-76 below, wherein the recovery breath detection and localization are performed by determining a first breath following a reduction in flow where the ventilation increases beyond a baseline ventilation.

56. The method according to 52 above or any or a combination of 1-55 above and/or 57-76 below, wherein the recovery breath detection and localization are performed by determining a first breath following a reduction in flow where a relative ventilation increases beyond 100%.

57. The method according to 38 above or any or a combination of 1-56 above and/or 58-76 below, further comprising quantifying a recovery breath amplitude based on the determined ventilation.

58. The method according to 57 above or any or a combination of 1-57 above and/or 59-76 below, wherein the recovery breath amplitude is an intended ventilation (i.e., respiratory drive) at arousal threshold.

59. The method according to 57 above or any or a combination of 1-58 above and/or 60-76 below, wherein the recovery breath amplitude is a ventilation at the recovery breath.

60. The method according to 57 above or any or a combination of 1-59 above and/or 61-76 below, wherein the recovery breath amplitude is an amplitude of the flow signal in the recovery

61. The method according to 57 above or any or a combination of 1-60 above and/or 62-76 below, wherein the recovery breath amplitude is a sum of a positive swing of the determined respiratory flow during a recovery breath.

62. The method according to any or a combination of 1-61 above and/or 63-76 below, further comprising:

determining ventilation based on the determined respiratory flow; and
quantifying a respiratory event depth in accordance with changes in the determined ventilation relative to a eupnea level.

63. The method according to 62 above or any or a combination of 1-62 above and/or 64-76 below, further comprising detecting respiratory event based on the quantified respiratory event depth.

64. The method according to 62 above or any or a combination of 1-63 above and/or 65-76 below, further comprising classifying a respiratory event as either apnea or hypopnea based on the quantified respiratory event depth.

65. The method according to 62 above or any or a combination of 1-64 above and/or 66-76 below, further comprising measuring an upper airway patency and/or measuring an upper airway collapsibility based on the quantified respiratory event depth.

66. The method according to any or a combination of 1-65 above and/or 67-76 below, further comprising:

determining ventilation or minute ventilation based on the determined respiratory flow, wherein the ventilation is one or more of
a tidal volume, which is a volume of air inhaled by the subject,
a volume of air inhaled by the person per minute,
a volume of air inhaled by the person per a predefined unit time,
a minute ventilation that is a sum of an inhalation of the respiratory flow,
a minute ventilation that is determined by integrating the inhalation of the respiratory flow,
a minute ventilation that is a sum of the inhalation of the respiratory flow that is divided by a duration of the inhalation, and/or
a flow signal originating from a flow sensor including an oro-nasal pneumotachograph, an oral pneumotachograph, a nasal pneumotachograph, a nasal cannula, an oral cannula, an oro-nasal cannula, and/or a respiratory inductance plethysmography.

67. The method according to any or a combination of 1-66 above and/or 68-76 below, further comprising:

determining ventilation based on the determined respiratory flow,
normalizing the ventilation based on a ventilation value for eupnea or normal breathing.

68. The method according to 67 above or any or a combination of 1-67 above and/or 69-76 below, wherein the normalizing of the ventilation is performed by

dividing the ventilation by a mean ventilation over a time period, wherein the time period is long enough so that the subject needs to maintain ventilation to support metabolism,
dividing the ventilation by the mean ventilation over the time period, wherein the time period is long enough so that the person needs to maintain ventilation to minimize or prevent the buildup of carbon dioxide in the blood,
dividing the ventilation by the mean ventilation over the time period, wherein the time period is long enough so that the subject needs to maintain ventilation to minimize a reduction in blood oxygen saturation,
dividing the ventilation by the mean ventilation over the time period, wherein the time period is in a range from 5 minutes to 10 minutes, or
dividing the ventilation by a surrogate signal that can be used as a proxy for metabolism.

69. The method according to any or a combination of 1-68 above and/or 70-76 below, further comprising:

determining ventilation based on the determined respiratory flow; and
determining respiratory event depth based on the determined ventilation, the respiratory event depth being determined by:
events are located using automatic scoring,
the respiratory event depth being a mean of a reduction of the ventilation during a respiratory event,
the respiratory event depth being a quantile between 5th percentile and a 25th percentile of the reduction of the ventilation during the respiratory event,
the respiratory event depth being a lowest ventilation during the respiratory event,
the respiratory event depth being the ventilation during a fraction of the respiratory event, or
the respiratory event depth being the ventilation during a period of predefined duration of the respiratory event for which the ventilation is lowest.

70. The method according to 2 above or any or a combination of 1-69 above and/or 71-76 below, wherein the one or more endotypes represent the physiological causes of sleep apnea.

71. The method according to 3 above or any or a combination of 1-70 above and/or 72-76 below, wherein the determining of the respiratory flow is performed by first applying the first calibration and then applying the third calibration.

72. The method according to 3 above or any or a combination of 1-71 above and/or 73-76 below, wherein the determining of the respiratory flow is performed by first applying the third calibration and then applying the first calibration.

73. The method according to 3 above or any or a combination of 1-72 above and/or 74-76 below, wherein

the second calibration is performed by applying a curve fit that outputs a multiplicative scaling based on an input of a correlation between the abdomen signal and the thoracic signal, the multiplicative scaling being multiplied with the abdomen signal and the thoracic signal or with a weighted sum of the abdomen signal and the thoracic signal, and
the curve fit being obtained by minimizing an objective function that relates the correlation between the abdomen signal and the thoracic signal to a ratio between the respiratory flow and a measured flow, which is obtained using a standard/validated flow measurement method.

74. The method according to 67 above or any or a combination of 1-73 above and/or 75-76 below, wherein the normalizing of the ventilation results in value of 100% for the ventilation corresponding to the eupnea or the normal breathing.

75. The method according to any or a combination of 1-74 above and/or 76 below, wherein receiving the data of the thoracic signal and receiving the data of the abdomen signal includes receiving the thoracic signal from the first RIP belt and the abdomen signal from the second RIP belt.

76. The method according to any or a combination of 1-75 above, wherein, in receiving the data of the thoracic signal and receiving the data of the abdomen signal, the data of the thoracic signal and the data of the abdomen signal are pre-recorded and stored in a storage medium and the data of the thoracic signal and the data of the abdomen signal are subsequently retrieved, transmitted, or received from the storage medium.

77. A system comprising:

a plurality of respiratory inductance plethysmography (RIP) belts, including a thoracic belt configured to measure a thoracic signal and a thoracic belt configured to measure an abdomen signal, wherein the thoracic signal corresponds to a length of the thoracic belt when arranged proximate with a thorax of a subject, and the abdomen signal corresponds to a length of the abdomen belt when arranged proximate with an abdomen of the subject; and
a processor configured to
receive the thoracic signal and the abdomen signal, and
determine a respiratory flow based on the thoracic signal and the abdomen signal, wherein the respiratory flow is determined using two or more calibrations, including:
a first calibration applying a first calibration coefficient that relates an amplitude of a differential change in the thoracic signal to an amplitude of a differential change in the abdomen signal, and
a second calibration that corrects for a non-linearity in the determined respiratory flow.

78. The system according to any or a combination of 77 above and/or 79-149 below, wherein the processor is further configured to determine one or more endotypes of an obstructive sleep apnea or of another sleep disorder of the subject based on the respiratory flow.

79. The system according to any or a combination of 77-78 above and/or 80-149 below, wherein the processor is further configured to determine the respiratory flow using a third calibration that corrects for an overestimation of flow during paradox.

80. The system according to any or a combination of 77-79 above and/or 81-149 below, wherein the thoracic signal represents a circumference of the thorax of the subject at a series of times, and the abdomen signal represents a circumference of the abdomen of the subject at the series of times.

81. The system according to any or a combination of 77-80 above and/or 82-149 below, wherein the processor is further configured to determine of the respiratory flow by calculating a change of the thoracic signal with respect to time to generate a derivative corresponding to a time derivative of a thoracic volume,

calculating a change of the abdomen signal with respect to time to generate another derivative corresponding to a time derivative of an abdomen volume, and
the respiratory flow is determined by combining the first derivative and the another derivative using a weighted sum that is based on the first calibration coefficient.
determining one or more endotypes of an obstructive sleep apnea or of another sleep disorder of the subject based on the respiratory flow.

82. The system according to any or a combination of 77-81 above and/or 83-149 below, wherein the processor is further configured to determine of the respiratory flow by calculating a time derivative of a calibrated sum of the thoracic signal and the abdomen signal, the calibrated sum being based on the first calibration coefficient.

83. The system according to any or a combination of 77-82 above and/or 84-149 below, wherein the first calibration is determined using the thoracic signal and the abdomen signal in an absence of all flow signals.

84. The system according to any or a combination of 77-83 above and/or 85-149 below, wherein the first calibration is determined using the thoracic signal and the abdomen signal in an absence of flow signals measured by one or more nasal canula.

85. The system according to any or a combination of 77-84 above and/or 86-149 below, wherein

the first calibration is carried out by selecting a value of the calibration coefficient that minimizes a function that represents a ratio of numerator to a denominator,
the numerator being a power of a weighted/scaled sum of the thoracic signal and the abdomen signal, wherein a weighting/scaling of the weighted/scaled sum is based on the value of the calibration coefficient,
the denominator being a power of the scaled thoracic signal summed with a power of a weighted/scaled abdomen signal, which is the abdomen signal that has been weighted/scaled sum is based on the value of the calibration coefficient, wherein
a weighting/scaling factor used to weight/scale the thoracic signal relative to the abdomen signal is the calibration coefficient or a function of the calibration coefficient.

86. The system according to any or a combination of 77-85 above and/or 87-149 below, wherein the first calibration is carried out by finding the value of k that satisfies to a predetermined threshold the equation

$${min}_{k} \frac{R M S ( ( 1 - k ) {dRIP}_{t h} + {kdRIP}_{a b} )}{R M S ( ( 1 - k ) {dRIP}_{th} ) + R M S ( {kdRIP}_{a b} )} ,$$

wherein RMS is the root mean square, k is a test value of the calibration coefficient, dRIPₜₕ represents a derivative of the thoracic signal with respect to time, dRIP_{ab} represents a derivative of the abdomen signal with respect to time.

87. The system according to any or a combination of 77-86 above and/or 88-149 below, wherein the first calibration is carried out using a PowerLoss calibration method.

88. The system according to 79 above or any or a combination of 77-87 above and/or 89-149 below, wherein the third calibration includes steps of

determining a correlation factor between the thoracic signal and the abdomen signal,
calculating an overestimation correction factor based on the correlation factor, and
the determining of the respiratory flow further includes scaling a derivative of a weighted sum of the thoracic signal and the abdomen signal by the overestimation correction factor.

89. The system according to 79 above or any or a combination of 77-88 above and/or 90-149 below, wherein the third calibration includes steps of

determining a correlation factor between the thoracic signal and the abdomen signal,
calculating an overestimation correction factor based on the correlation factor, and
the determining of the respiratory flow further includes scaling the thoracic signal and the abdomen signal by the overestimation correction factor.

90. The system according to any or a combination of 77-89 above and/or 91-149 below, wherein the second calibration includes steps of

calculating a non-linearity correction factor based on a curve fit, the curve fit having been generated from calibration data that includes the respiratory flow, which is uncorrected for the non-linearity, and a reference flow, which is measured using trusted/validated method and is acquired concurrently with the respiratory flow of the calibration data, and
the determining of the respiratory flow further includes scaling a derivative of a weighted sum of the thoracic signal and the abdomen signal by the non-linearity correction factor.

91. The system according to any or a combination of 77-90 above and/or 92-149 below, wherein the second calibration includes steps of

calculating a non-linearity correction factor based on a curve fit, the curve fit having been generated from calibration data that includes the respiratory flow, which is uncorrected for the non-linearity, and a reference flow, which is measured using trusted/validated method and is acquired concurrently with the respiratory flow of the calibration data, and
the determining of the respiratory flow further includes scaling the thoracic signal and the abdomen signal by the non-linearity correction factor.

92. The system according to 88 above or any or a combination of 77-91 above and/or 93-149 below, wherein the curve fit for the calculating of the non-linearity correction factor has an exponential functional form and the non-linearity correction factor is an exponent.

93. The system according to 89 above or any or a combination of 77-92 above and/or 94-149 below, wherein the curve fit for the calculating of the non-linearity correction factor has an exponential functional form and the non-linearity correction factor is an exponent.

94. The system according to any or a combination of 77-93 above and/or 95-149 below, wherein the processor is further configured to determine the respiratory flow by first applying the first calibration and then applying the second calibration.

95. The system according to any or a combination of 77-94 above and/or 96-149 below, wherein the processor is further configured to determine of the respiratory flow by first applying the second calibration and then applying the first calibration.

96. The system according to 79 above or any or a combination of 77-95 above and/or 97-149 below, wherein the processor is further configured to determine the respiratory flow by first applying the second calibration and then applying the third calibration.

97. The system according to 79 above or any or a combination of 77-96 above and/or 98-149 below, wherein the processor is further configured to determine the respiratory flow by first applying the third calibration and then applying the second calibration.

98. The system according to 79 above or any or a combination of 77-97 above and/or 99-149 below, wherein the processor is further configured to determine the respiratory flow by first applying the second calibration, then applying the third calibration, and then applying the first calibration.

99. The system according to 79 above or any or a combination of 77-98 above and/or 100-149 below, wherein the processor is further configured to determine the respiratory flow by first applying the second calibration, then applying the first calibration, and then applying the third calibration.

100. The system according to 79 above or any or a combination of 77-99 above and/or 101-149 below, wherein the processor is further configured to determine the respiratory flow by first applying the third calibration, then applying the second calibration, and then applying the first calibration.

101. The system according to 79 above or any or a combination of 77-100 above and/or 102-149 below, wherein the processor is further configured to determine the respiratory flow by first applying the first calibration, then applying the second calibration, and then applying the third calibration.

102. The system according to 79 above or any or a combination of 77-101 above and/or 103-149 below, wherein the processor is further configured to determine the respiratory flow by first applying the first calibration, then applying the third calibration, and then applying the second calibration.

103. The system according to any or a combination of 77-102 above and/or 104-149 below, wherein the processor is further configured determine periods of constant calibration by detecting changes a first DC signal, which is DC offset component of the thoracic signal, and/or a second DC signal, which is DC offset component of the abdomen signal, and indicating that periods between the detected changes are respectively periods of constant calibration, and recalibrate the first calibration within each of the periods of constant calibration based on the thoracic signal and the abdomen signal within the respective periods of constant calibration.

104. The system according to 103 above or any or a combination of 77-103 above and/or 105-149 below, wherein, during a given period of the periods of constant calibration, the recalibrating of the first calibration includes obtaining a value of the first calibration coefficient that minimizes a ventilation that would be obtained from the thoracic signal and the abdomen signal during the paradox within the given period.

105. The system according to any or a combination of 77-104 above and/or 106-149 below, wherein the processor is further configured to determine the respiratory flow without relying on direct measurement of oral breathing.

106. The system according to any or a combination of 77-105 above and/or 107-149 below, wherein the determining of the respiratory flow is performed without relying on direct measurement of nasal breathing.

107. The system according to any or a combination of 77-106 above and/or 108-149 below, wherein the processor is further configured to determine the respiratory flow without relying on direct measurement of oral breathing and without relying on direct measurement of nasal breathing.

108. The system according to any or a combination of 77-107 above and/or 109-149 below, wherein the processor is further configured to determine ventilation based on the thoracic signal and the abdomen signal.

109. The system according to any or a combination of 77-108 above and/or 110-149 below, wherein the processor is further configured to determine ventilation based on the thoracic signal and the determined respiratory flow.

110. The system according to 78 above or any or a combination of 77-109 above and/or 111-149 below, wherein the processor is further configured to determine the one or more endotypes by determining an endotype including upper airway collapsibility (Vpassive), upper airway muscle compensation (Vcomp), ventilation at the arousal threshold (Vactive), arousal threshold, and/or loop gain.

111. The system according to 78 above or any or a combination of 77-110 above and/or 112-149 below, wherein the processor is further configured to treat or prescribe a treatment for the subject based on the determined one or more endotypes.

112. The system according to 79 above or any or a combination of 77-111 above and/or 113-149 below, wherein the third calibration includes a curve fit that is personalized to the subject.

113. The system according to 78 above or any or a combination of 77-112 above and/or 114-149 below, wherein the processor is further configured to determine ventilation based on the determined respiratory flow; and quantify relative changes in the determined ventilation and therefrom and detecting one or more respiratory events.

114. The system according to 113 above or any or a combination of 77-113 above and/or 115-149 below, wherein the processor is further configured to detect the one or more respiratory events, wherein the one or more respiratory events include apneas, hypopneas, flow restriction, flow limitation, snoring, obstructed breathing, normal breathing, and/or eupnea.

115. The system according to 113 above or any or a combination of 77-114 above and/or 116-149 below, wherein the processor is further configured to localize the one or more respiratory events with respect to time.

116. The system according to 115 above or any or a combination of 77-115 above and/or 117-149 below, wherein the one or more respiratory events is hypoxic burden.

117. The system according to 115 above or any or a combination of 77-116 above and/or 118-149 below, wherein the processor is further configured to use the localized the one or more respiratory events to improve an accuracy of manual event scoring.

118. The system according to 115 above or any or a combination of 77-117 above and/or 119-149 below, wherein the processor is further configured to use the localized one or more respiratory events to improve event duration estimates.

119. The system according to 115 above or any or a combination of 77-118 above and/or 120-149 below, wherein the processor is further configured to localize the one or more respiratory events by determining two points in time, a first point in time being a start time of a respiratory event and a second point being a stop time of the respiratory event, and the first point being determined by a predefined reduction in ventilation and the second point being determined by a recovery of ventilation.

120. The system according to 113 above or any or a combination of 77-119 above and/or 121-149 below, wherein the processor is further configured determine an event severity for the one or more respiratory events.

121. The system according to 120 above or any or a combination of 77-120 above and/or 122-149 below, wherein the event severity is a measure of upper airway patency and/or collapsibility.

122. The system according to 120 above or any or a combination of 77-121 above and/or 123-149 below, wherein the processor is further configured classify respiratory events into apnea or hypopnea based on the event severity.

123. The system according to 120 above or any or a combination of 77-122 above and/or 124-149 below, wherein the event severity is determined by an event duration as measured by a time difference between a first point in time and a second point in time, the first point in time being a start time of a respiratory event and the second point being a stop time of the respiratory event, and the first point being determined by a predefined reduction in ventilation and the second point being determined by a recovery of ventilation.

124. The system according to 120 above or any or a combination of 77-123 above and/or 125-149 below, wherein the event severity is determined by and event depth, the event depth being a relative reduction in ventilation.

125. The system according to 120 above or any or a combination of 77-124 above and/or 126-149 below, wherein the event severity is determined by an apnea burden, the apnea burden being the cumulative loss of ventilation during the event.

126. The system according to 125 above or any or a combination of 77-125 above and/or 127-149 below, wherein the processor is further configured to calculate the cumulative loss of ventilation by one of

integrating a reduction in ventilation for the event,
summing a difference between a baseline ventilation and a reduced ventilation during the event,
determining a baseline ventilation as the ventilation at normal breathing, or
determining the baseline ventilation as the mean ventilation in a time window where the time window is long enough to require the person to maintain ventilation to sustain metabolism.

127. The system according to 113 above or any or a combination of 77-126 above and/or 128-149 below, wherein the one or more respiratory events is a recovery breath, and the processor is further configured to localize the recovery breath with respect to time.

128. The system according to 127 above or any or a combination of 77-127 above and/or 129-149 below, wherein the processor is further configured to use the recovery breath detection and localization to detect accurate respiratory arousal.

129. The system according to 127 above or any or a combination of 77-128 above and/or 130-149 below, wherein the processor is further configured to use the recovery breath detection and localization to determine a first breath after the one or more respiratory events.

130. The system according to 127 above or any or a combination of 77-129 above and/or 131-149 below, wherein the processor is further configured to detect and localize the recovery breath by determining a first breath following a reduction in flow where the ventilation increases beyond a baseline ventilation.

131. The system according to 127 above or any or a combination of 77-130 above and/or 132-149 below, wherein the processor is further configured to detect and localize the recovery breath detection and localization by determining a first breath following a reduction in flow where a relative ventilation increases beyond 100%.

132. The system according to any or a combination of 77-131 above and/or 133-149 below, wherein the processor is further configured to quantify a recovery breath amplitude based on the determined ventilation.

133. The system according to 113 above or any or a combination of 77-132 above and/or 134-149 below, wherein the processor is further configured to determine the recovery breath amplitude, wherein the recovery breath amplitude is an intended ventilation (i.e., respiratory drive) at arousal threshold.

134. The system according to 113 above or any or a combination of 77-133 above and/or 135-149 below, wherein the processor is further configured to determine the recovery breath amplitude, wherein the recovery breath amplitude is a ventilation at the recovery breath.

135. The system according to 113 above or any or a combination of 77-134 above and/or 136-149 below, wherein the processor is further configured to determine the recovery breath amplitude, wherein the recovery breath amplitude is an amplitude of the flow signal in the recovery

136. The system according to 113 above or any or a combination of 77-135 above and/or 137-149 below, wherein the processor is further configured to determine the recovery breath amplitude, wherein the recovery breath amplitude is a sum of a positive swing of the determined respiratory flow during a recovery breath.

137. The system according to any or a combination of 77-136 above and/or 138-149 below, wherein the processor is further configured to

determine ventilation based on the determined respiratory flow; and
quantify a respiratory event depth in accordance with changes in the determined ventilation relative to a eupnea level.

138. The system according to 137 above or any or a combination of 77-137 above and/or 139-149 below, wherein the processor is further configured to detect a respiratory event based on the quantified respiratory event depth.

139. The system according to 137 above or any or a combination of 77-138 above and/or 140-149 below, wherein the processor is further configured to classify a respiratory event as either apnea or hypopnea based on the quantified respiratory event depth.

140. The system according to 137 above or any or a combination of 77-139 above and/or 141-149 below, wherein the processor is further configured to determine an upper airway patency and/or determine an upper airway collapsibility based on the quantified respiratory event depth.

141. The system according to any or a combination of 77-140 above and/or 142-149 below, wherein the processor is further configured to

determine ventilation or minute ventilation based on the determined respiratory flow, wherein the ventilation is one of
a tidal volume, which is a volume of air inhaled by the subject,
a volume of air inhaled by the person per minute,
a volume of air inhaled by the person per a predefined unit time,
a minute ventilation that is a sum of an inhalation of the respiratory flow,
a minute ventilation that is determined by integrating the inhalation of the respiratory flow,
a minute ventilation that is a sum of the inhalation of the respiratory flow that is divided by a duration of the inhalation.
a flow signal originating from a flow sensor including an oro-nasal pneumotachograph, an oral pneumotachograph, a nasal pneumotachograph, a nasal cannula, an oral cannula, an oro-nasal cannula, and/or a respiratory inductance plethysmography.

142. The system according to any or a combination of 77-141 above and/or 143-149 below, wherein the processor is further configured to determine ventilation based on the determined respiratory flow, and normalize the ventilation based on a ventilation value for eupnea or normal breathing.

143. The system according to 142 above or any or a combination of 77-142 above and/or 144-149 below, wherein the normalizing of the ventilation results in value of 100% for the ventilation corresponding to the eupnea or the normal breathing.

144. The system according to 142 above or any or a combination of 77-143 above and/or 145-149 below, wherein the normalizing of the ventilation is performed by dividing the ventilation by a mean ventilation over a time period, wherein the time period is long enough so that the subject needs to maintain ventilation to support metabolism,

dividing the ventilation by the mean ventilation over the time period, wherein the time period is long enough so that the person needs to maintain ventilation to minimize or prevent the buildup of carbon dioxide in the blood,
dividing the ventilation by the mean ventilation over the time period, wherein the time period is long enough so that the subject needs to maintain ventilation to minimize a reduction in blood oxygen saturation,
dividing the ventilation by the mean ventilation over the time period, wherein the time period is in a range from 5 minutes to 10 minutes, or
dividing the ventilation by a surrogate signal that can be used as a proxy for metabolism.

145. The system according to 79 above or any or a combination of 77-144 above and/or 146-149 below, wherein the processor is further configured to determine the respiratory flow by first applying the first calibration and then applying the third calibration.

146. The system according to 79 above or any or a combination of 77-145 above and/or 147-149 below, wherein the processor is further configured to determine the respiratory flow by first applying the third calibration and then applying the first calibration.

147. The system according to 78 above or any or a combination of 77-146 above and/or 148-149 below, wherein the one or more endotypes represent the physiological causes of sleep apnea.

148. The system according to 79 above or any or a combination of 77-147 above and/or 149 below, wherein

the second calibration is performed by applying a curve fit that outputs a multiplicative scaling based on an input of a correlation between the abdomen signal and the thoracic signal, the multiplicative scaling being multiplied with the abdomen signal and the thoracic signal or with a weighted sum of the abdomen signal and the thoracic signal, and
the curve fit being obtained by minimizing an objective function that relates the correlation between the abdomen signal and the thoracic signal to a ratio between the respiratory flow and a measured flow, which is obtained using a standard/validated flow measurement method.

149. The system according to any or a combination of 77-148 above, wherein the processor is further configured to

determine ventilation based on the determined respiratory flow; and
determine a respiratory event depth based on the determined ventilation, the respiratory event depth being determined by:
events are located using automatic scoring,
the respiratory event depth being a mean of a reduction of the ventilation during a respiratory event,
the respiratory event depth being a quantile between 5th percentile and a 25th percentile of the reduction of the ventilation during the respiratory event,
the respiratory event depth being a lowest ventilation during the respiratory event,
the respiratory event depth being the ventilation during a fraction of the respiratory event, or
the respiratory event depth being the ventilation during a period of predefined duration of the respiratory event for which the ventilation is lowest.

150. A computer readable medium having stored thereon instructions that, when executed by a processor, cause the processor to execute the steps of method according to any or a combination of 1-76 above for determining a respiratory flow from respiratory inductance plethysmography (RIP) signals, including:

receiving data of a thoracic signal that corresponds to a length of a first RIP belt arranged proximate with a thorax of a subject;
receiving data of an abdomen signal that corresponds to a length of a second RIP belt arranged proximate with an abdomen of the subject; and
determining a respiratory flow based on the thoracic signal and the abdomen signal, wherein the respiratory flow is determined using two or more calibrations, including:
a first calibration applying a first calibration coefficient that relates an amplitude of a differential change in the thoracic signal to an amplitude of a differential change in the abdomen signal, and
a second calibration that corrects for a non-linearity in the determined respiratory flow.

151. A device for determining a respiratory flow from respiratory inductance plethysmography (RIP) signals, the device comprising:

a processor configured to
receive a thoracic signal that corresponds to a length of a first RIP belt arranged proximate with a thorax of a subject;
receive an abdomen signal that corresponds to a length of a second RIP belt arranged proximate with an abdomen of the subject; and
determine a respiratory flow based on the thoracic signal and the abdomen signal, wherein the respiratory flow is determined using two or more calibrations, including:
a first calibration applying a first calibration coefficient that relates an amplitude of a differential change in the thoracic signal to an amplitude of a differential change in the abdomen signal, and
a second calibration that corrects for a non-linearity in the determined respiratory flow.

152. The device according to any or a combination of 151 above and/or 153-223 below, wherein the processor is further configured to determine one or more endotypes of an obstructive sleep apnea or of another sleep disorder of the subject based on the respiratory flow.

153. The device according to any or a combination of 151-152 above and/or 154-223 below, wherein the processor is further configured to determine the respiratory flow using a third calibration that corrects for an overestimation of flow during paradox.

154. The device according to any or a combination of 151-153 above and/or 155-223 below, wherein the thoracic signal represents a circumference of the thorax of the subject at a series of times, and the abdomen signal represents a circumference of the abdomen of the subject at the series of times.

155. The device according to any or a combination of 151-154 above and/or 156-223 below, wherein the processor is further configured to determine of the respiratory flow by calculating a change of the thoracic signal with respect to time to generate a derivative corresponding to a time derivative of a thoracic volume,

calculating a change of the abdomen signal with respect to time to generate another derivative corresponding to a time derivative of an abdomen volume, and
the respiratory flow is determined by combining the first derivative and the another derivative using a weighted sum that is based on the first calibration coefficient.
determining one or more endotypes of an obstructive sleep apnea or of another sleep disorder of the subject based on the respiratory flow.

156. The device according to any or a combination of 151-155 above and/or 157-223 below, wherein the processor is further configured to determine of the respiratory flow by calculating a time derivative of a calibrated sum of the thoracic signal and the abdomen signal, the calibrated sum being based on the first calibration coefficient.

157. The device according to any or a combination of 151-156 above and/or 158-223 below, wherein the first calibration is determined using the thoracic signal and the abdomen signal in an absence of all flow signals.

158. The device according to any or a combination of 151-157 above and/or 159-223 below, wherein the first calibration is determined using the thoracic signal and the abdomen signal in an absence of flow signals measured by one or more nasal canula.

159. The device according to any or a combination of 151-158 above and/or 160-223 below, wherein

the first calibration is carried out by selecting a value of the calibration coefficient that minimizes a function that represents a ratio of numerator to a denominator,
the numerator being a power of a weighted/scaled sum of the thoracic signal and the abdomen signal, wherein a weighting/scaling of the weighted/scaled sum is based on the value of the calibration coefficient,
the denominator being a power of the scaled thoracic signal summed with a power of a weighted/scaled abdomen signal, which is the abdomen signal that has been weighted/scaled sum is based on the value of the calibration coefficient, wherein
a weighting/scaling factor used to weight/scale the thoracic signal relative to the abdomen signal is the calibration coefficient or a function of the calibration coefficient.

160. The device according to any or a combination of 151-159 above and/or 161-223 below, wherein the first calibration is carried out by finding the value of k that satisfies to a predetermined threshold the equation

$${min}_{k} \frac{R M S ( ( 1 - k ) {dRIP}_{t h} + {kdRIP}_{a b} )}{R M S ( ( 1 - k ) {dRIP}_{th} ) + R M S ( {kdRIP}_{a b} )} ,$$

wherein RMS is the root mean square, k is a test value of the calibration coefficient, dRIPₜₕ represents a derivative of the thoracic signal with respect to time, dRIP_{ab} represents a derivative of the abdomen signal with respect to time.

161. The device according to any or a combination of 151-160 above and/or 162-223 below, wherein the first calibration is carried out using a PowerLoss calibration method.

162. The device according to 153 above or any or a combination of 151-161 above and/or 163-223 below, wherein the third calibration includes steps of

determining a correlation factor between the thoracic signal and the abdomen signal,
calculating an overestimation correction factor based on the correlation factor, and
the determining of the respiratory flow further includes scaling a derivative of a weighted sum of the thoracic signal and the abdomen signal by the overestimation correction factor.

163. The device according to 153 above or any or a combination of 151-162 above and/or 164-223 below, wherein the third calibration includes steps of

determining a correlation factor between the thoracic signal and the abdomen signal,
calculating an overestimation correction factor based on the correlation factor, and
the determining of the respiratory flow further includes scaling the thoracic signal and the abdomen signal by the overestimation correction factor.

164. The device according to any or a combination of 151-163 above and/or 165-223 below, wherein the second calibration includes steps of

calculating a non-linearity correction factor based on a curve fit, the curve fit having been generated from calibration data that includes the respiratory flow, which is uncorrected for the non-linearity, and a reference flow, which is measured using trusted/validated method and is acquired concurrently with the respiratory flow of the calibration data, and
the determining of the respiratory flow further includes scaling a derivative of a weighted sum of the thoracic signal and the abdomen signal by the non-linearity correction factor.

165. The device according to any or a combination of 151-164 above and/or 166-223 below, wherein the second calibration includes steps of

calculating a non-linearity correction factor based on a curve fit, the curve fit having been generated from calibration data that includes the respiratory flow, which is uncorrected for the non-linearity, and a reference flow, which is measured using trusted/validated method and is acquired concurrently with the respiratory flow of the calibration data, and
the determining of the respiratory flow further includes scaling the thoracic signal and the abdomen signal by the non-linearity correction factor.

166. The device according to 162 above or any or a combination of 151-165 above and/or 167-223 below, wherein the curve fit for the calculating of the non-linearity correction factor has an exponential functional form and the non-linearity correction factor is an exponent.

167. The device according to 163 above or any or a combination of 151-166 above and/or 168-223 below, wherein the curve fit for the calculating of the non-linearity correction factor has an exponential functional form and the non-linearity correction factor is an exponent.

168. The device according to any or a combination of 151-167 above and/or 169-223 below, wherein the processor is further configured to determine the respiratory flow by first applying the first calibration and then applying the second calibration.

169. The device according to any or a combination of 151-168 above and/or 170-223 below, wherein the processor is further configured to determine of the respiratory flow by first applying the second calibration and then applying the first calibration.

170. The device according to 153 above or any or a combination of 151-169 above and/or 171-223 below, wherein the processor is further configured to determine the respiratory flow by first applying the second calibration and then applying the third calibration.

171. The device according to 153 above or any or a combination of 151-170 above and/or 172-223 below, wherein the processor is further configured to determine the respiratory flow by first applying the third calibration and then applying the second calibration.

172. The device according to 153 above or any or a combination of 151-171 above and/or 173-223 below, wherein the processor is further configured to determine the respiratory flow by first applying the second calibration, then applying the third calibration, and then applying the first calibration.

173. The device according to 153 above or any or a combination of 151-172 above and/or 174-223 below, wherein the processor is further configured to determine the respiratory flow by first applying the second calibration, then applying the first calibration, and then applying the third calibration.

174. The device according to 153 above or any or a combination of 151-173 above and/or 175-223 below, wherein the processor is further configured to determine the respiratory flow by first applying the third calibration, then applying the second calibration, and then applying the first calibration.

175. The device according to 153 above or any or a combination of 151-174 above and/or 176-223 below, wherein the processor is further configured to determine the respiratory flow by first applying the first calibration, then applying the second calibration, and then applying the third calibration.

176. The device according to 153 above or any or a combination of 151-175 above and/or 177-223 below, wherein the processor is further configured to determine the respiratory flow by first applying the first calibration, then applying the third calibration, and then applying the second calibration.

177. The device according to any or a combination of 151-176 above and/or 178-223 below, wherein the processor is further configured determine periods of constant calibration by detecting changes a first DC signal, which is DC offset component of the thoracic signal, and/or a second DC signal, which is DC offset component of the abdomen signal, and indicating that periods between the detected changes are respectively periods of constant calibration, and recalibrate the first calibration within each of the periods of constant calibration based on the thoracic signal and the abdomen signal within the respective periods of constant calibration.

178. The device according to 177 above or any or a combination of 151-177 above and/or 179-223 below, wherein, during a given period of the periods of constant calibration, the recalibrating of the first calibration includes obtaining a value of the first calibration coefficient that minimizes a ventilation that would be obtained from the thoracic signal and the abdomen signal during the paradox within the given period.

179. The device according to any or a combination of 151-178 above and/or 180-223 below, wherein the processor is further configured to determine the respiratory flow without relying on direct measurement of oral breathing.

180. The device according to any or a combination of 151-179 above and/or 181-223 below, wherein the determining of the respiratory flow is performed without relying on direct measurement of nasal breathing.

181. The device according to any or a combination of 151-180 above and/or 182-223 below, wherein the processor is further configured to determine the respiratory flow without relying on direct measurement of oral breathing and without relying on direct measurement of nasal breathing.

182. The device according to any or a combination of 151-181 above and/or 183-223 below, wherein the processor is further configured to determine ventilation based on the thoracic signal and the abdomen signal.

183. The device according to any or a combination of 151-182 above and/or 184-223 below, wherein the processor is further configured to determine ventilation based on the thoracic signal and the determined respiratory flow.

184. The device according to 152 above or any or a combination of 151-183 above and/or 185-223 below, wherein the processor is further configured to determine the one or more endotypes by determining an endotype including upper airway collapsibility (Vpassive), upper airway muscle compensation (Vcomp), ventilation at the arousal threshold (Vactive), arousal threshold, and/or loop gain.

185. The device according to 152 above or any or a combination of 151-184 above and/or 186-223 below, wherein the processor is further configured to treat or prescribe a treatment of the subject based on the determined one or more endotypes.

186. The device according to 153 above or any or a combination of 151-185 above and/or 187-223 below, wherein the third calibration includes a curve fit that is personalized to the subject.

187. The device according to 153 above or any or a combination of 151-186 above and/or 188-223 below, wherein the processor is further configured to determine ventilation based on the determined respiratory flow; and quantify relative changes in the determined ventilation and therefrom and detecting one or more respiratory events.

188. The device according to 187 above or any or a combination of 151-187 above and/or 189-223 below, wherein the processor is further configured to detect the one or more respiratory events, wherein the one or more respiratory events include apneas, hypopneas, flow restriction, flow limitation, snoring, obstructed breathing, normal breathing, and/or eupnea.

189. The device according to 187 above or any or a combination of 151-188 above and/or 190-223 below, wherein the processor is further configured to localize the one or more respiratory events with respect to time.

190. The device according to 189 above or any or a combination of 151-189 above and/or 191-223 below, wherein the one or more respiratory events is hypoxic burden.

191. The device according to 189 above or any or a combination of 151-190 above and/or 192-223 below, wherein the processor is further configured to use the localized the one or more respiratory events to improve an accuracy of manual event scoring.

192. The device according to 189 above or any or a combination of 151-191 above and/or 193-223 below, wherein the processor is further configured to use the localized one or more respiratory events to improve event duration estimates.

193. The device according to 189 above or any or a combination of 151-192 above and/or 194-223 below, wherein the processor is further configured to localize the one or more respiratory events by determining two points in time, a first point in time being a start time of a respiratory event and a second point being a stop time of the respiratory event, and the first point being determined by a predefined reduction in ventilation and the second point being determined by a recovery of ventilation.

194. The device according to 187 above or any or a combination of 151-193 above and/or 195-223 below, wherein the processor is further configured determine an event severity for the one or more respiratory events.

195. The device according to 194 above or any or a combination of 151-194 above and/or 196-223 below, wherein the event severity is a measure of upper airway patency and/or collapsibility.

196. The device according to 194 above or any or a combination of 151-195 above and/or 197-223 below, wherein the processor is further configured classify respiratory events into apnea or hypopnea based on the event severity.

197. The device according to 194 above or any or a combination of 151-196 above and/or 198-223 below, wherein the event severity is determined by an event duration as measured by a time difference between a first point in time and a second point in time, the first point in time being a start time of a respiratory event and the second point being a stop time of the respiratory event, and the first point being determined by a predefined reduction in ventilation and the second point being determined by a recovery of ventilation.

198. The device according to 194 above or any or a combination of 151-197 above and/or 199-223 below, wherein the event severity is determined by and event depth, the event depth being a relative reduction in ventilation.

199. The device according to 194 above or any or a combination of 151-198 above and/or 200-223 below, wherein the event severity is determined by an apnea burden, the apnea burden being the cumulative loss of ventilation during the event.

200. The device according to 199 above or any or a combination of 151-199 above and/or 201-223 below, wherein the processor is further configured to calculate the cumulative loss of ventilation by one of

integrating a reduction in ventilation for the event,
summing a difference between a baseline ventilation and a reduced ventilation during the event,
determining a baseline ventilation as the ventilation at normal breathing, or
determining the baseline ventilation as the mean ventilation in a time window where the time window is long enough to require the person to maintain ventilation to sustain metabolism.

201. The device according to 187 above or any or a combination of 151-200 above and/or 202-223 below, wherein the one or more respiratory events is a recovery breath, and the processor is further configured to localize the recovery breath with respect to time.

202. The device according to 201 above or any or a combination of 151-201 above and/or 203-223 below, wherein the processor is further configured to use the recovery breath detection and localization to detect accurate respiratory arousal.

203. The device according to 201 above or any or a combination of 151-202 above and/or 204-223 below, wherein the processor is further configured to use the recovery breath detection and localization to determine a first breath after the one or more respiratory events.

204. The device according to 201 above or any or a combination of 151-203 above and/or 205-223 below, wherein the processor is further configured to detect and localize the recovery breath by determining a first breath following a reduction in flow where the ventilation increases beyond a baseline ventilation.

205. The device according to 201 above or any or a combination of 151-204 above and/or 206-223 below, wherein the processor is further configured to detect and localize the recovery breath detection and localization by determining a first breath following a reduction in flow where a relative ventilation increases beyond 100%.

206. The device according to 187 above or any or a combination of 151-205 above and/or 207-223 below, wherein the processor is further configured to determine quantify a recovery breath amplitude based on the determined ventilation.

207. The device according to 206 above or any or a combination of 151-206 above and/or 208-223 below, wherein the processor is further configured to determine the recovery breath amplitude, wherein the recovery breath amplitude is an intended ventilation (i.e., respiratory drive) at arousal threshold.

208. The device according to 206 above or any or a combination of 151-207 above and/or 209-223 below, wherein the processor is further configured to determine the recovery breath amplitude, wherein the recovery breath amplitude is a ventilation at the recovery breath.

209. The device according to 206 above or any or a combination of 151-208 above and/or 210-223 below, wherein the processor is further configured to determine the recovery breath amplitude, wherein the recovery breath amplitude is an amplitude of the flow signal in the recovery

210. The device according to 206 above or any or a combination of 151-209 above and/or 211-223 below, wherein the processor is further configured to determine the recovery breath amplitude, wherein the recovery breath amplitude is a sum of a positive swing of the determined respiratory flow during a recovery breath.

211. The device according to any or a combination of 151-210 above and/or 212-223 below, wherein the processor is further configured to

determine ventilation based on the determined respiratory flow; and
quantify a respiratory event depth in accordance with changes in the determined ventilation relative to a eupnea level.

212. The device according to 211 above or any or a combination of 151-211 above and/or 213-223 below, wherein the processor is further configured to detect a respiratory event based on the quantified respiratory event depth.

213. The device according to 211 above or any or a combination of 151-212 above and/or 214-223 below, wherein the processor is further configured to classify a respiratory event as either apnea or hypopnea based on the quantified respiratory event depth.

214. The device according to 211 above or any or a combination of 151-213 above and/or 215-223 below, wherein the processor is further configured to determine an upper airway patency and/or determine an upper airway collapsibility based on the quantified respiratory event depth.

215. The device according to any or a combination of 151-214 above and/or 216-223 below, wherein the processor is further configured to

determine ventilation or minute ventilation based on the determined respiratory flow, wherein the ventilation is one of
a tidal volume, which is a volume of air inhaled by the subject,
a volume of air inhaled by the person per minute,
a volume of air inhaled by the person per a predefined unit time,
a minute ventilation that is a sum of an inhalation of the respiratory flow,
a minute ventilation that is determined by integrating the inhalation of the respiratory flow,
a minute ventilation that is a sum of the inhalation of the respiratory flow that is divided by a duration of the inhalation.
a flow signal originating from a flow sensor including an oro-nasal pneumotachograph, an oral pneumotachograph, a nasal pneumotachograph, a nasal cannula, an oral cannula, an oro-nasal cannula, and/or a respiratory inductance plethysmography.

216. The device according to any or a combination of 151-215 above and/or 217-223 below, wherein the processor is further configured to determine ventilation based on the determined respiratory flow, and normalize the ventilation based on a ventilation value for eupnea or normal breathing.

217. The device according to 216 above or any or a combination of 151-216 above and/or 218-223 below, wherein the normalizing of the ventilation results in value of 100% for the ventilation corresponding to the eupnea or the normal breathing.

218. The device according to 216 above or any or a combination of 151-217 above and/or 219-223 below, wherein the normalizing of the ventilation is performed by

dividing the ventilation by a mean ventilation over a time period, wherein the time period is long enough so that the subject needs to maintain ventilation to support metabolism,
dividing the ventilation by the mean ventilation over the time period, wherein the time period is long enough so that the person needs to maintain ventilation to minimize or prevent the buildup of carbon dioxide in the blood,
dividing the ventilation by the mean ventilation over the time period, wherein the time period is long enough so that the subject needs to maintain ventilation to minimize a reduction in blood oxygen saturation,
dividing the ventilation by the mean ventilation over the time period, wherein the time period is in a range from 5 minutes to 10 minutes, or
dividing the ventilation by a surrogate signal that can be used as a proxy for metabolism.

219. The device according to 153 above or any or a combination of 151-218 above and/or 220-223 below, wherein the processor is further configured to determine the respiratory flow by first applying the first calibration and then applying the third calibration.

220. The device according to 153 above or any or a combination of 151-219 above and/or 221-223 below, wherein the processor is further configured to determine the respiratory flow by first applying the third calibration and then applying the first calibration.

221. The device according to 152 above or any or a combination of 151-220 above and/or 222-223 below, wherein the one or more endotypes represent the physiological causes of sleep apnea.

222. The device according to 153 above or any or a combination of 151-221 above and/or 223-223 below, wherein

the third calibration is performed by applying a curve fit that outputs a multiplicative scaling based on an input of a correlation between the abdomen signal and the thoracic signal, the multiplicative scaling being multiplied with the abdomen signal and the thoracic signal or with a weighted sum of the abdomen signal and the thoracic signal, and
the curve fit being obtained by minimizing an objective function that relates the correlation between the abdomen signal and the thoracic signal to a ratio between the respiratory flow and a measured flow, which is obtained using a standard/validated flow measurement method.

223. The device according to any or a combination of 151-152 above, wherein the processor is further configured to

determine ventilation based on the determined respiratory flow; and
determine a respiratory event depth based on the determined ventilation, the respiratory event depth being determined by:
events are located using automatic scoring,
the respiratory event depth being a mean of a reduction of the ventilation during a respiratory event,
the respiratory event depth being a quantile between 5th percentile and a 25th percentile of the reduction of the ventilation during the respiratory event,
the respiratory event depth being a lowest ventilation during the respiratory event,
the respiratory event depth being the ventilation during a fraction of the respiratory event, or
the respiratory event depth being the ventilation during a period of predefined duration of the respiratory event for which the ventilation is lowest.

224. A computer readable medium having stored thereon instructions that, when executed by a processor, cause the processor to execute steps for determining a respiratory flow from respiratory inductance plethysmography (RIP) signals, the steps comprising of a method according to any one or a combination of 1-76 above.

225. A method for determining a respiratory flow from respiratory inductance plethysmography (RIP) signals, the method comprising:

receiving data of a thoracic signal that corresponds to a length of a first RIP belt arranged proximate with a thorax of a subject;
receiving data of an abdomen signal that corresponds to a length of a second RIP belt arranged proximate with an abdomen of the subject; and
determining a respiratory flow based on the thoracic signal and the abdomen signal, wherein the respiratory flow is determined using one or more calibrations, including:
calibration (a) that includes applying a first calibration coefficient that relates an amplitude of a differential change in the thoracic signal to an amplitude of a differential change in the abdomen signal,
calibration (b) that corrects for a non-linearity in the determined respiratory flow, or
calibration (c) that corrects for an overestimation of flow during paradox.

226. The method according to any or a combination of 225 above or 227-244 below, wherein the respiratory flow is determined using

calibration (a) that includes applying a first calibration coefficient that relates an amplitude of a differential change in the thoracic signal to an amplitude of a differential change in the abdomen signal.

227. The method according to any or a combination of 225-226 above or 228-244 below, wherein the respiratory flow is determined using

calibration (b) that corrects for a non-linearity in the determined respiratory flow.

228. The method according to any or a combination of 225-227 above or 229-244 below, wherein the respiratory flow is determined using

calibration (c) that corrects for an overestimation of flow during paradox.

229. The method according to any or a combination of 225-228 above or 230-244 below, wherein the respiratory flow is determined using

calibration (a) that includes applying a first calibration coefficient that relates an amplitude of a differential change in the thoracic signal to an amplitude of a differential change in the abdomen signal,
and
calibration (b) that corrects for a non-linearity in the determined respiratory flow.

230. The method according to any or a combination of 225-229 above or 231-244 below, wherein the respiratory flow is determined using

calibration (a) that includes applying a first calibration coefficient that relates an amplitude of a differential change in the thoracic signal to an amplitude of a differential change in the abdomen signal,
and subsequently
calibration (b) that corrects for a non-linearity in the determined respiratory flow.

231. The method according to any or a combination of 225-230 above or 232-244 below, wherein the respiratory flow is determined using

calibration (b) that corrects for a non-linearity in the determined respiratory flow
and subsequently
calibration (a) that includes applying a first calibration coefficient that relates an amplitude of a differential change in the thoracic signal to an amplitude of a differential change in the abdomen signal.

232. The method according to any or a combination of 225-231 above or 233-244 below, wherein the respiratory flow is determined using

calibration (a) that includes applying a first calibration coefficient that relates an amplitude of a differential change in the thoracic signal to an amplitude of a differential change in the abdomen signal,
and
calibration (c) that corrects for an overestimation of flow during paradox.

233. The method according to any or a combination of 225-232 above or 234-244 below, wherein the respiratory flow is determined using

calibration (a) that includes applying a first calibration coefficient that relates an amplitude of a differential change in the thoracic signal to an amplitude of a differential change in the abdomen signal,
and subsequently
calibration (c) that corrects for an overestimation of flow during paradox.

234. The method according to any or a combination of 225-233 above or 235-244 below, wherein the respiratory flow is determined using

calibration (c) that corrects for an overestimation of flow during paradox,
and subsequently
calibration (a) that includes applying a first calibration coefficient that relates an amplitude of a differential change in the thoracic signal to an amplitude of a differential change in the abdomen signal.

235. The method according to any or a combination of 225-234 above or 236-244 below, wherein the respiratory flow is determined using

calibration (b) that corrects for a non-linearity in the determined respiratory flow,
and
calibration (c) that corrects for an overestimation of flow during paradox.

236. The method according to any or a combination of 225-235 above or 237-244 below, wherein the respiratory flow is determined using

calibration (b) that corrects for a non-linearity in the determined respiratory flow,
and subsequently
calibration (c) that corrects for an overestimation of flow during paradox.

237. The method according to any or a combination of 225-236 above or 238-244 below, wherein the respiratory flow is determined using

calibration (c) that corrects for an overestimation of flow during paradox,
and subsequently
calibration (b) that corrects for a non-linearity in the determined respiratory flow.

238. The method according to any or a combination of 225-237 above or 239-244 below, wherein the respiratory flow is determined using

calibration (a) that includes applying a first calibration coefficient that relates an amplitude of a differential change in the thoracic signal to an amplitude of a differential change in the abdomen signal,
calibration (b) that corrects for a non-linearity in the determined respiratory flow, and
calibration (c) that corrects for an overestimation of flow during paradox.

239. The method according to any or a combination of 225-238 above or 240-244 below, wherein the respiratory flow is determined using

calibration (a) that includes applying a first calibration coefficient that relates an amplitude of a differential change in the thoracic signal to an amplitude of a differential change in the abdomen signal,
and subsequently
calibration (b) that corrects for a non-linearity in the determined respiratory flow,
and subsequently
calibration (c) that corrects for an overestimation of flow during paradox.

240. The method according to any or a combination of 225-239 above or 241-244 below, wherein the respiratory flow is determined using

calibration (a) that includes applying a first calibration coefficient that relates an amplitude of a differential change in the thoracic signal to an amplitude of a differential change in the abdomen signal,
and subsequently
calibration (c) that corrects for an overestimation of flow during paradox,
and subsequently
calibration (b) that corrects for a non-linearity in the determined respiratory flow.

241. The method according to any or a combination of 225-240 above or 242-244 below, wherein the respiratory flow is determined using

calibration (b) that corrects for a non-linearity in the determined respiratory flow,
and subsequently
calibration (a) that includes applying a first calibration coefficient that relates an amplitude of a differential change in the thoracic signal to an amplitude of a differential change in the abdomen signal,
and subsequently
calibration (c) that corrects for an overestimation of flow during paradox.

242. The method according to any or a combination of 225-241 above or 243-244 below, wherein the respiratory flow is determined using

calibration (b) that corrects for a non-linearity in the determined respiratory flow,
and subsequently
calibration (c) that corrects for an overestimation of flow during paradox,
and subsequently
calibration (a) that includes applying a first calibration coefficient that relates an amplitude of a differential change in the thoracic signal to an amplitude of a differential change in the abdomen signal.

243. The method according to any or a combination of 225-242 above or 244 below, wherein the respiratory flow is determined using

calibration (c) that corrects for an overestimation of flow during paradox, and subsequently
calibration (a) that includes applying a first calibration coefficient that relates an amplitude of a differential change in the thoracic signal to an amplitude of a differential change in the abdomen signal,
and subsequently
calibration (b) that corrects for a non-linearity in the determined respiratory flow.

244. The method according to 225-226 above of 227-244 below, wherein the respiratory flow is determined using

calibration (c) that corrects for an overestimation of flow during paradox,
and subsequently
calibration (b) that corrects for a non-linearity in the determined respiratory flow,
and subsequently
calibration (a) that includes applying a first calibration coefficient that relates an amplitude of a differential change in the thoracic signal to an amplitude of a differential change in the abdomen signal.

245. A computer readable medium having stored thereon instructions that, when executed by a processor, cause the processor to execute steps for determining a respiratory flow from respiratory inductance plethysmography (RIP) signals, the steps comprising of a method according to any one or a combination of 225 through 244 above.

246. A system comprising:

a plurality of respiratory inductance plethysmography (RIP) belts, including a thoracic belt configured to measure a thoracic signal and a thoracic belt configured to measure an abdomen signal, wherein the thoracic signal corresponds to a length of the thoracic belt when arranged proximate with a thorax of a subject, and the abdomen signal corresponds to a length of the abdomen belt when arranged proximate with an abdomen of the subject; and
a processor configured to perform the method according to any one or a combination of 225 through 244 above.

247. A device for determining a respiratory flow from respiratory inductance plethysmography (RIP) signals, the device comprising:

a processor configured to perform the method according to any one or a combination of 225 through 244 above.

1. A method for determining a respiratory flow from data from respiratory inductance plethysmography (RIP) signals, the method comprising:
receiving data of a thoracic signal of a first RIP belt arranged proximate with a thorax of a subject;
receiving data of an abdomen signal of a second RIP belt arranged proximate with an abdomen of the subject; and
determining a respiratory flow of the subject based on the data of the thoracic signal and the data of the abdomen signal,
wherein determining the respiratory flow includes two or more calibrations, including:
performing a first calibration by applying a first calibration coefficient that relates an amplitude of a differential change in the thoracic signal to an amplitude of a differential change in the abdomen signal to obtain a determined respiratory flow, and
performing a second calibration on the determined respiratory flow that corrects for a non-linearity in the determined respiratory flow.

2. The method according to claim 1, further comprising a step of determining one or more endotypes of an obstructive sleep apnea or of another sleep disorder of the subject based on the determined, calibrated respiratory flow.

3. The method according to claim 1, wherein determining the respiratory flow further includes a third calibration that corrects for an overestimation of flow during paradox.

4. The method according to claim 1, wherein the method further comprises taking respective derivatives of the thoracic signal and the abdomen signal and combining the derivatives using a scaling coefficient to determine the respiratory flow.

5. The method according to claim 1, wherein determining of the respiratory flow includes
calculating a change of the thoracic signal with respect to time to generate a derivative corresponding to a time derivative of a thoracic volume,
calculating a change of the abdomen signal with respect to time to generate another derivative corresponding to a time derivative of an abdomen volume, and
the respiratory flow is determined by combining the derivative and the another derivative using a weighted sum that is based on the first calibration coefficient.

6. The method according to claim 1, wherein the determining of the respiratory flow includes calculating a time derivative of a calibrated sum of the thoracic signal and the abdomen signal, the calibrated sum being based on the first calibration coefficient.

7. The method according to claim 1, wherein the first calibration is determined using the thoracic signal and the abdomen signal in an absence of all flow signals.

8. The method according to claim 1, wherein the first calibration is determined using the thoracic signal and the abdomen signal in an absence of flow signals measured by one or more nasal canula.

9. The method according to claim 1, wherein
the first calibration is carried out by selecting a value of the calibration coefficient that minimizes a function that represents a ratio of numerator to a denominator,
the numerator being a power of a weighted/scaled sum of the thoracic signal and the abdomen signal, wherein a weighting/scaling of the weighted/scaled sum is based on the value of the calibration coefficient,
the denominator being a power of the scaled thoracic signal summed with a power of a weighted/scaled abdomen signal, which is the abdomen signal that has been weighted/scaled sum is based on the value of the calibration coefficient,
wherein a weighting/scaling factor used to weight/scale the thoracic signal relative to the abdomen signal is the calibration coefficient or a function of the calibration coefficient.

10. The method according to claim 1, wherein the first calibration is carried out by finding the value of k that satisfies to a predetermined threshold the equation
$${min}_{k} \frac{R M S ( ( 1 - k ) {dRIP}_{t h} + {kdRIP}_{a b} )}{R M S ( ( 1 - k ) {dRIP}_{th} ) + R M S ( {kdRIP}_{a b} )} ,$$
wherein RMS is the root mean square, k is a test value of the calibration coefficient, dRIPₜₕ represents a derivative of the thoracic signal with respect to time, dRIP_{ab} represents a derivative of the abdomen signal with respect to time.

11. The method according to claim 1, wherein the first calibration is carried out using a PowerLoss calibration method.

12. The method according to claim 3, wherein the third calibration includes steps of
determining a correlation factor between the thoracic signal and the abdomen signal,
calculating an overestimation correction factor based on the correlation factor, and
the determining of the respiratory flow further includes scaling a derivative of a weighted sum of the thoracic signal and the abdomen signal by the overestimation correction factor.

13. The method according to claim 3, wherein the third calibration includes steps of
determining a correlation factor between the thoracic signal and the abdomen signal,
calculating an overestimation correction factor based on the correlation factor, and
the determining of the respiratory flow further includes scaling the thoracic signal and the abdomen signal by the overestimation correction factor.

14. The method according to claim 1, wherein the second calibration includes steps of
calculating a non-linearity correction factor based on a curve fit, the curve fit having been generated from calibration data that includes the respiratory flow, which is uncorrected for the non-linearity, and a reference flow, which is measured using trusted/validated method and is acquired concurrently with the respiratory flow of the calibration data, and
the determining of the respiratory flow further includes scaling a derivative of a weighted sum of the thoracic signal and the abdomen signal by the non-linearity correction factor.

15. The method according to claim 1, wherein the second calibration includes steps of
calculating a non-linearity correction factor based on a curve fit, the curve fit having been generated from calibration data that includes the respiratory flow, which is uncorrected for the non-linearity, and a reference flow, which is measured using trusted/validated method and is acquired concurrently with the respiratory flow of the calibration data, and
the determining of the respiratory flow further includes scaling the thoracic signal and the abdomen signal by the non-linearity correction factor.

16. The method according to claim 15, wherein the curve fit for the calculating of the non-linearity correction factor has an exponential functional form and the non-linearity correction factor is an exponent.

17. The method according to claim 16, wherein the curve fit for the calculating of the non-linearity correction factor has an exponential functional form and the non-linearity correction factor is an exponent.

18. A computer readable medium having stored thereon instructions that, when executed by one or more processors of a computing system cause the one or more processors to execute the steps of the method according to claim 1.

19. A device for determining a respiratory flow from data from respiratory inductance plethysmography (RIP) signals, the device comprising:
a processor configured to
receive data of a thoracic signal of a first RIP belt arranged proximate with a thorax of a subject;
receive data of an abdomen signal of a second RIP belt arranged proximate with an abdomen of the subject; and
determine a respiratory flow of the subject based on the data of the thoracic signal and the data of the abdomen signal,
wherein determining the respiratory flow includes two or more calibrations, includes:
performing a first calibration by applying a first calibration coefficient that relates an amplitude of a differential change in the thoracic signal to an amplitude of a differential change in the abdomen signal to obtain a determined respiratory flow, and
performing a second calibration on the determined respiratory flow that corrects for a non-linearity in the determined respiratory flow.

20. A system comprising:
a plurality of respiratory inductance plethysmography (RIP) belts, including a thoracic belt configured to obtain a thoracic signal and an abdomen belt configured to obtain an abdomen signal; and
a processor configured to
receive data of the thoracic signal of a first RIP belt arranged proximate with a thorax of a subject;
receive data of the abdomen signal of a second RIP belt arranged proximate with an abdomen of the subject; and
determine a respiratory flow of the subject based on the data of the thoracic signal and the data of the abdomen signal,
wherein determining the respiratory flow includes two or more calibrations, includes:
performing a first calibration by applying a first calibration coefficient that relates an amplitude of a differential change in the thoracic signal to an amplitude of a differential change in the abdomen signal to obtain a determined respiratory flow, and
performing a second calibration on the determined respiratory flow that corrects for a non-linearity in the determined respiratory flow.
